(19) United States
(12) Patent Application Publication
(10) Pub. No.: US 2013/0165468 A1
(43) Pub. Date: Jun. 27, 2013

(54) TOPICAL PERIPHERAL NEURO-AFFECTIVE (TPNA) THERAPY

(71) Applicant: Ronald Aung-Din, Sarasota, FL (US)

(72) Inventor: Ronald Aung-Din, Sarasota, FL (US)

(73) Assignee: AFGIN PHARMA, LLC, Sarasota, FL (US)

(21) Appl. No.: 13/697,501

(22) PCT Filed: May 13, 2011
(86) PCT No.: PCT/US11/36394

§ 371 Date: Mar. 12, 2013

Related U.S. Application Data
(60) Provisional application No. 61/334,325, filed on May 13, 2010.

(51) Int. Cl. A61K 31/485; A61K 31/433; A61K 31/135; A61K 31/473

(52) U.S. Cl. CPC A61K 31/485; A61K 31/473; A61K 31/433; A61K 31/135
USPC 514/282; 514/284; 514/362; 514/646

(57) ABSTRACT

A method of treating peripheral neuropathic pain in humans resulting from a peripheral nerve injury and for treating muscle spasm in humans resulting from a peripheral nerve injury comprises applying a therapeutically effective amount of a drug selected from the group consisting of a dopamine agonist, a skeletal muscle relaxant, and a combination thereof topically to the site of the injury.

### FIELD OF THE INVENTION

The invention relates to topical peripheral neuro-affective therapy (“TRNA THERAPY”) for the treatment of peripheral neuropathic pain and muscle spasm: peripheral nerve and neuronal hyperexcitability and neurochemical dysfunction syndromes.

### BACKGROUND OF THE INVENTION

Topical Regional Neuro-Affective (TRNA, and also known as Nuchal Topical Neuro-Afferent Therapeutics, NT) therapy using “Central Nervous System (CNS)-active” drugs for the treatment of brain electro-chemical dysfunctional states has been previously described. The inventor's previous U.S. Patent Publication No. 20030013753 (filed Jun. 5, 2002) and U.S. Patent Publication No. 20080090894, both of which are hereby incorporated by reference, disclose a unit dose of a topical formulation for treating a migraine or cluster headache comprising: a serotonin agonist incorporated into a pharmaceutically acceptable vehicle for topical administration onto the skin, specifically at the back of neck/nape at the hairline (BONATH) of a human patient. This is in order to capitalize on the relationship that exists at this site with respect to the cutaneous free nerve-endings and the afferent nerves relaying back to the Central Nervous System, not found elsewhere. Preferably, the unit dose providing the serotonin agonist is in a form that is immediately absorbable when said unit dose is applied onto human skin. Preferably, the serotonin agonist comprises from about 0.5 to about 200 mg of sumatriptan, by weight based on the succinate salt, or a therapeutic equivalent dose of another topically absorbable pharmaceutically acceptable serotonin agonist. Preferably, the unit dose provides relief from a migraine or cluster headache within about 2 hours after topical administration to a human patient.

The inventor's previous U.S. Patent Publication No. 20070065463 (filed Jun. 21, 2004) discloses a topical formulation for treating migraines or cluster headaches, muscle sprains, muscle spasms, spasticity, tension headaches, tension related migraines and related conditions associated with muscle tension and pain comprising: a therapeutically effective amount of an active agent(s) incorporated into a pharmaceutically acceptable excipient for topical administration onto the skin of a human patient, the active agent(s) being selected from the group consisting of: i) an ergot alkaloid; ii) a skeletal muscle relaxant; or iii) a combination of an ergot alkaloid and a skeletal muscle relaxant; the active agent(s) being present in an effective concentration such that a unit dose of the topical formulation provides a therapeutic effect within about 2 hours after topical administration to the human patient. In certain preferred embodiments, the topical formulation comprises a skeletal muscle relaxant such as tizanidine. In certain preferred embodiments, the unit dose comprises from about 0.4 mg to 8 mg, preferably from about 0.2 mg to about 4 mg of tizanidine hydrochloride.

In these methods, active drug compounded in an appropriate “dermal penetration-enhancing medium” is applied at the back of the neck, the nape or nuchal region, at the hairline (“BONATH”), capitalizing on the specific relationship of the cutaneous free nerve-endings at this location with the afferent neural systems of the trigeminal, vagal, and sympathetic nerves with the CNS. By affecting afferent neural input (nerve impulses returning from the periphery/body to the brain), CNS efferent neural output (nerve impulses generated by the brain and outgoing to the body), as clinical symptoms, is modulated. Depending on the specific neuroanatomy and neurochemical processes involved in the pathologic or dysfunctional state, clinical symptoms may vary in presentation. With migraine, they are headache, nausea, visual obscurations, and dizziness as the trigeminal nerve and the serotonergic systems are involved.

Alternatively, in Parkinson' disease (PD), the dopaminergic system within the nigra-striatal pathways is affected. The clinical symptoms in this case are tremor, rigidity, postural instability, and; bradykinesia and bradyphrenia (slowed movements and thought processes), Muscle pain and depression are also common.

The inventor's previously filed International Patent Application WO 2010/005507 discloses a method of treating a disease state or condition in humans with a drug comprising administering a drug selected from the group consisting of anti-epileptic, an anxiolytic, a neuroleptic, an anti-psychotic, an analgesic, an anti-inflammatory, an anti-Parkinson's disease/syndrome drug, a sexual dysfunction drug, a drug for the treatment of dystonia, a drug for the treatment of spastic conditions, a drug for the treatment of benign essential/familial tremor, a drug for the treatment of tremor, a drug for the treatment of chronic encepahalopathies, a drug for the treatment of congenital CNS degeneration conditions/cerebral palsy, a drug for the treatment of cerebellar degeneration syndromes, a drug for the treatment of neuropathic and/or neurogenic pain, a drug for smoking cessation, a drug for appetite suppression, a drug for neurodegenerative conditions, a drug for the treatment of multiple sclerosis, a drug for the treatment of insomnia, a drug for the treatment of fatigue, a drug for the treatment of vertigo, nausea and/or dizziness, a drug for the treatment of writer's cramp and restless leg syndrome, a drug for the treatment of ADD/ADHD, in a therapeutically effective amount to treat the disease state or condition, to the back of the neck at the hairline in close proximity to and under or on the area of skin above the brain stem to provide regional neuro-affective therapy to the patient. Thus, TRNA drug delivery, applied at the back of the neck at the hairline (“BONATH”) may potentially be used for any disease state which has as its basis a dysfunctional electro-chemical state within the brain. Besides migraine and PD, other conditions shown amenable to TRNA therapy include: tension headache, tremor of various etiologies, spastic conditions from brain injury, seizure disorders; and mood disorders such as depression, anxiety, psychotic states, and bi-polar affective disorder. Also included are other brain-derived disorders such as erectile dysfunction, restless legs syndrome, OCD and compulsive behaviors such as gambling, addictions, and hypersexual states. These latter conditions involve dysfunction within the dopamine neurochemical system.

Most skeletal muscle relaxants are centrally acting and are administered via the oral route or parenteral (by injection) route. The drawback of the oral or parenteral administration is that there are frequent systemic side effects such as fatigue, lethargy, weakness and mental clouding, particularly as higher doses are reached. Benzodiazepines, e.g., diazepam, have additional drawbacks such as tolerance, psychological dependency and withdrawal effects, e.g., seizures. Oral administration route also entails delay of drug effect through gastrointestinal absorption and systemic circulation.

In certain instances skeletal muscle relaxants can also be administered topically. For example, U.S. Pat. No. 5,364,628 to Kissel et al. describes a transdermal adhesive plaster or patch containing tizanidine for application every three days for the systemic treatment of rheumatic pains and muscle spasms. Also, UK Patent Application No. 2098865 to Joachim Franz et al. describes a composition and method for administering a sustained release micro emulsion containing tizanidine. A suitable dose of 10-50 mg of tizanidine may be administered, which provides an effect for up to three (3) days. Although topical administration has been described in the art, FDA approval has only been granted for oral and parenteral administration of skeletal muscle relaxants.

### SUMMARY OF THE INVENTION

It is an object of the present invention to provide a method of treatment in humans with topical brainstem deafferentation therapy via the regional administration of a compound useful for the treatment of such diseases or conditions that may be treated via such therapy.

It is an object of the present invention to provide a method for the treatment of peripheral neuropathic pain and muscle spasm, including but not limited to peripheral nerve and neuronal hyperexcitability and neurochemical dysfunction syndromes.

It is a further object of the present invention to provide a method for the treatment of acute sports injuries and those of chronic nature such as fibromyalgia, complex regional pain syndrome/reflex sympathetic dystrophy, neuralgias, nerve entrapment syndromes, chronic musculo-skeletal pain from osteoarthritis, degenerative spinal disc/joint disease, and the like.

In accordance with the above objects and others, the invention is directed in part to a method of treating peripheral neuropathic pain in humans resulting from a peripheral nerve injury comprising applying a therapeutically effective amount of a drug selected from the group consisting of a dopamine agonist, a skeletal muscle relaxant, an opioid agonist, and any combination thereof at the site of the injury, e.g., topically to the site of the injury.

The present invention is further directed in part to a method of treating pain in humans resulting from acute sports injuries and injuries of a chronic nature such as fibromyalgia, complex regional pain syndrome/reflex sympathetic dystrophy, neuralgias, nerve entrapment syndromes, chronic musculo-skeletal pain from osteoarthritis, degenerative spinal disc/joint disease, and the like via the administration of an opioid agonist at the site of the injury, e.g., topically to the site of the injury.

The invention is further directed in part to the invention is directed in part to a method of treating muscle spasm in humans resulting from a peripheral nerve injury comprising applying a therapeutically effective amount of a drug selected from the group consisting of a dopamine agonist, a skeletal muscle relaxant, an opioid agonist, an SNRI, and any combination thereof topically to the site of the injury. In certain preferred embodiments, the injury is neuronal hyperexcitability and/or a neurochemical dysfunction syndrome.

Certain embodiments of the present invention are directed to a method of treating generalized and/or isolated peripheral pain conditions by applying a therapeutically effective amount of a drug selected from the group consisting of a dopamine agonist, a skeletal muscle relaxant, an opioid agonist, an SNRI, and any combination thereof at two or more sites along the nerve, preferably concurrently. In certain preferred embodiments, a therapeutically effective amount of the drug(s) is also applied to the nuchal area, preferably concurrently. With regard to applications along a nerve, it is contemplated herein that this aspect of the method is useful where major “named” nerves are concerned with obvious locations that are accessible to treatment. For example, for the Median or Ulnar Nerve at the wrist (carpal tunnnel), arm, and elbow—cubital tunnel. For the Tibial Nerve at the ankle: “tarsal tunnel”. For the Peroneal Nerve, further application at the fibula head and at the popliteal fossa at the knee. These examples are not meant to be exclusive and are made simply for the purposes of explanation. In situations where there has been injury, the method encompasses application of the drug(s) at the site of the injury (e.g., topical application at that site), as well as local nerve's respective entry points in the spine—lumbar for the lower extremity and the cervical spine for upper. Additionally, the Nuchal region is used as the last point of drug effect along the nerve route returning to the CNS. Thus, aspects of the present invention further contemplate two or three application sites, depending on the nerve affected and the place of injury. The drug (same or different drug) is applied as two or three full doses or one-two divided doses at the application sites.

For purposes of the present invention, a “topical formulation” includes, for example, ointments, creams, lotions, pastes, gels, etc., which releases one or more drugs (e.g., dopamine agonists) at a predetermined rate over a defined period of time to a defined site of application.

For purposes of the present invention, an “injectable” formulation includes, for example, an injectable solution, suspension, gel or the like and may be in immediate release form or may provide a controlled or sustained release of the drug at the site of administration.

For purposes of the present invention, an “implantable” formulation includes, for example, a solid, semisolid or liquid drug formulation which can be administered at the site of administration (e.g., BONATH) either via injection and/or via surgical implantation. The solid may comprise microspheres, microcapsules, pellets, discs, and the like. The implantable formulations of the invention may provide a controlled or sustained release of the drug at the site of administration.

For purposes of the present invention, a “transdermal therapeutic system” is defined as a drug-containing device (including e.g., patch, disc, etc.) which releases one or more drugs at a predetermined rate over a defined period of time to a defined site of application.

For purposes of the present invention, “transdermal” delivery is the delivery by passage of a drug through the skin and into the bloodstream (“traditional” transdermal delivery) and is termed “transdermal systemic drug delivery (TSD therapy).

For purposes of the present invention, the term “topical transdermal therapy” is synonomous with the more accurately termed topical regional neuro-affective therapy (or “TRNA therapy”). The delivery process is also referred to herein as Nuchal Topical Neuro-Afferent Therapeutics. This term describes important aspects of this delivery method: topical, regional (near brainstem and cervical spinal cord), and affecting the free nerve endings of the afferent nervous system through drug application at the nape or nuchal region of the neck.

For purposes of the present invention “therapeutically effective” or “effective” amount is meant to be a nontoxic but sufficient amount of a compound to provide the desired therapeutic effect, e.g., avoidance of the onset of a migraine and or increased alleviation of the migraine and/or cluster headache. In the present case, for example, it is the dose of serotonin agonist that will be effective in relieving symptoms of the migraine or cluster headache. An “effective” amount of a permeation enhancer as used herein, for example, means an amount that will provide the desired increase in skin permeability and, correspondingly, the desired depth of penetration, rate of administration, and amount of drug to be delivered.

For purposes of the present invention, the term “delivers” when used with respect to the topical formulation or transdermal therapeutic system means that the formulation or system provides a mean relative release rate or flux of the drug out of the formulation or system and through the skin of the patient.

By “predetermined area of skin” is intended a defined area of intact unbroken living skin. In certain embodiments of the present invention, the predetermined area will be in the range of about 1 cm2 to about 100 cm2, preferably in the range of about 10 cm2 to about 100 cm2, more preferably in the range of about 20 cm2 to about 60 cm2. However, it will be appreciated by those skilled in the art of topical delivery that the area of skin through which drug is administered may vary significantly, depending on the formulation, dose, the application of the formulation, and the like.

“Penetration enhancement” or “permeation enhancement” for purposes of the present invention relates to an increase in the permeability of skin to a pharmacologically active agent, i.e., so as to increase the rate at which the drug permeates through the skin and enters the bloodstream. The enhanced permeation effected through the use of such enhancers can be observed by measuring the rate of diffusion of drug through animal or human skin using a diffusion cell apparatus.

For purposes of the present invention, the “brainstem afferent stimulation therapy region” is defined as the skin region of the head and/or at the frontotemporal region and/or upper posterior cervical area. In certain preferred embodiments, the treatment area is the post cervical area in close proximity to the brain stem. Preferably this area is a relatively hairless area of the patient's head and/or neck.

For purposes of the present invention, the phrase “to the site of the injury” means that the pharmaceutical formulation including the drug is applied topically or administered via injection directly at the injured structure or in proximity thereto.

For purposes of the present invention, the drug may be in the form of the base, or may be provided as a pharmaceutically acceptable salt (inorganic or organic) or complex. It may be in an optically pure form or a mixture of stereoisomers.

### DETAILED DESCRIPTION

The present invention is directed in part to the fact that TRNA may be applied for peripheral nerve injury states with associated pain and muscle spasm. The symptoms may be from localized phenomena in addition to that of afferent feed-back to CNS sensory processing areas with efferent outflow back to the site of injury and other regions.

Generally the drug applications have been over areas of nerve entrapment (tarsal and carpal tunnel syndromes) or over the affected muscle and its insertion points. From the above observations, it is apparent TRNA may be applied for peripheral nerve injury states with associated pain and muscle spasm. The symptoms may be from localized phenomena in addition to that of afferent feed-back to CNS sensory processing areas with efferent outflow back to the site of injury and other regions

The drug formulations useful in the present invention may be in a form selected from a topical formulation (e.g, a cream, ointment or gel); a transdermal device; or an implantable or injectable formulation.

In certain preferred embodiments, the active agent(s) is included in a topical formulation further comprising one or more pharmaceutically acceptable excipients that aid in the absorption of the active agent(s) when a unit dose of the formulation is applied topically to the headache region of the human patient.

In certain other embodiments, the active agent(s) provide a localized effect and/or reduced side effects.

The premise by which the topical application of a neuro-active drug at the BONATH relieves clinical symptoms of these CNS disorders rests on the fact that any brain-derived electro-chemical dysfunctional state is reflected neurochemically (and therefore, electrochemically) at the skin free nerve-ending level. Some skin areas are more affected than others, depending on the condition involved. For example, the “cutaneous allodynia” perceived by migraineurs some time before the onset of a migraine headache attack represents such phenomena. Cutaneous allodynia is described as tingling, uncomfortable heightened sensations involving the scalp, face, back of the neck; and sometimes, the extremities, such as the fingers. Descriptions have included “hair painful to touch.” These symptoms are attributed to the activation of the trigeminal nerve system at the onset of the migraine process. The cutaneous free nerve-endings of the skin of the head, face, and neck supplied by the trigeminal nerve are activated before the formal migraine attack and are responsible for these symptoms. Through the network of the trigeminal nerve with the cervical nerves and nerve roots, the vagus nerve, and the cervical sympathetic system within the soft tissues of the neck; nausea, upset stomach, emotional irritability, and sensory symptoms of the hands may occur as these systems also become involved.

Another common example of the continuous feed-back loop existing between the CNS and its peripherally innervated areas (of which the cutaneous free nerve-endings are end components), is localized phenomena at the back of the neck (BONATH) during periods of severe psychological agitation. In such episodes, one may experience the sensation of “the hair at the back of the neck standing up.” This may be accompanied by posterior cervical muscle spasm. The affected individual may describe the stress-inducing situation as “a pain in the neck.” Tingling sensations of the fingertips with enhanced physiologic tremor may occur. Other autonomic symptoms of flushing, increased heart rate and blood pressure, and bowel and bladder symptoms are also commonly encountered. These allude to the extensive neural network between the skin at the back of neck (BONATH) and the autonomic nerves with the CNS.

### Relationship Between the CNS and Cutaneous Free Nerve Endings

The neural intimacy between CNS and skin free nerve-endings exists in that both brain and skin are derived from the same embryonic tissue, neuro-ectoderm. The relationship is necessary for survival as the skin serves as the sentinel to injury from the outside world. With even a minor injury, as a “paper cut” or a pin-prick to the finger, neural impulses are immediately transmitted through afferent pathways to the brain as part of the pain response.

The corresponding efferent outflow from CNS results in a reflex “withdrawal response” from the pain inducing agent. The pain response has with it varying degrees of autonomic and associated psychological reactions; such as sweating, clamminess of the extremities, and irritability. Sensations of feeling faint or actual passing out may occur as the result of excessive vagal nerve activation, the “vaso-vagal response.” The exact response observed depends on the offending stimulus as well as the individual's physiological and psychological makeup as it relates to pain perception. For example, in a severe “needle-phobic”, the response to a needle stick can be particularly dramatic.

When active drug (generally a neurochemical modulator), is topically applied to the skin at the BONATH, it acts on the free nerve-endings at this location where aspects of a CNS electrochemical dysfunctional state is represented at the cutaneous free nerve ending receptors as part of the neural feedback loop. The inventor has previously reported that neurochemical activation by topically applied drug at the free nerve-endings (at the BONATH) is transmitted as afferent neural impulses to CNS. This results in brain efferent neural outflow modulation, the effect of which may be resolution or relief of clinical symptoms. That symptom relief, regardless of the treated disease state with TRNA drug delivery, is generally noted within 5-10 minutes of application suggests neural pathways are operating in the therapeutic process without involvement of the bloodstream. The relatively low doses of drug required for therapeutic benefit and the lack of systemic side effects further support this hypothesis.

The efferent (or descending from CNS) as well as the afferent (or ascending to the CNS) pathways which make up the neural reflex loop may both be influenced by drugs which act to either stimulate or inhibit their respective functions. In this regard, drugs used in TRNA therapy may be specifically selected to accomplish the desired effects on these pathways to affect clinical symptoms.

### Clinical Experience with TRNA at BONATH

Clinically, TRNA drug delivery at BONATH has been successfully used in migraine, tension headache, cervical spasm, spasticity, tremors of various causes (essential, cerebellar, and PD related); and other aspects of PD—rigidity, bradykinesia, bradyphrenia, speech and swallow problems, and gait difficulty. Other conditions where clinical efficacy has been demonstrated include erectile dysfunction, pathologic gambling, and mood disorders; particularly, anxiety states. To date the drug classes used in TRNA have been the triptans (serotonin agonists), the muscle relaxant tizanidine (adrenergic agonist); and apomorphine, the combination dopamine and adrenergic agonist.

The triptans and tizanidine are used “prn,” or as needed, for conditions with episodic symptoms, such as headache and muscle spasms. TRNA apomorphine, on the other hand, is used in conditions like PD, tremor, and other movement disorders with continuous symptoms. TRNA apomorphine for the specific treatment of erectile dysfunction (ED) is used on “as needed” basis.

### TRNA for Peripheral Neuropathic Pain

In the present invention, TRNA principles as discussed above have been applied to localized peripheral neuropathic pain (as opposed to CNS-derived conditions previously discussed).

Generally the drug applications have been over areas of nerve entrapment (tarsal and carpal tunnel syndromes) or over the affected muscle and its insertion points. From the above observations, it is apparent TRNA may be applied for peripheral nerve injury states with associated pain and muscle spasm. The symptoms may be from localized phenomena in addition to that of afferent feed-back to CNS sensory processing areas with efferent outflow back to the site of injury and other regions.

Localized peripheral neuropathic pain may be caused by conditions such as the following: traumatic injuries to the body affecting soft tissue and bone where associated nerve injury or irritation has also taken place to cause pain with or without muscle spasm. These may consist of burns, bruising, fractures, nerve impingement syndromes (carpal and tarsal tunnel syndromes, cubital tunnel, peripheral neuropathies), as well as bites and infections with skin eruptions such as occur with post-herpetic neuralgia. Muscle and ligamentous stretch injuries, as occur in sports are included.

Certain embodiments of the invention are directed to a method of treatment, comprising delivering a drug(s) through regional neuro-affective therapy by application as a cream/gel or a sustained release patch applied at the site of the injury, or via administration under the skin at the site of the injury via an implantable or injectable drug formulation or device.

In certain embodiments, the method further provides for a therapeutically effective treatment through transdermal regional neuro-affective (TRNA) therapy by application of a drug(s) as a cream/gel or a sustained release patch applied at the site of the injury.

The present peripheral TRNA delivery differs from traditional therapy (whether oral, injection, nasal spray, inhalation, or rectal) in that it has no reliance on the systemic or cerebral blood flow. Nor does it require therapeutic blood levels of drug. These latter factors are responsible for systemic and CNS side-effects as drug is delivered to areas not intended to be affected in the therapeutic process. Transdermal systemic delivery by patch, although similarly applied to the skin as in TRNA BONATH therapy, differs significantly in its reliance on a drug concentration gradient for absorption into the systemic capillary and venous blood. TRNA therapy is unaffected by dermal vessels or systemic blood flow. It relies solely on the function of the free nerve endings of cutaneous nerves and their connections at the point of application of compounded drug.

“Traditional” transdermal drug delivery by patch and TRNA are both “transdermal” in that in both, drug penetrates the skin (epidermis) for eventual clinical effect. The difference lies in the fact that in “traditional” transdermal patch therapy, drug enters the systemic circulation through a concentration gradient and establishes a therapeutic drug blood level. Although measuring a blood level gives assurance drug is being taken or delivered systemically, allowing for checking compliance, it is also the source of undesirable side-effects and drug interactions. Of necessity, with systemic transdermal patch therapy, drug applied to the skin surface must be absorbed through the small vessels in the dermis for eventual presence in the systemic venous blood for measurement of drug level. With the proposed TRNA therapy, drug need only be available at the free nerve endings under the epidermis. No concentration gradients or systemic blood levels are necessary. Drug delivery is unaffected by cardiac output or cerebral blood flow factors. Of significance, persons afflicted with Parkinson's disease are typically elderly with concomitant cardiac and cerebral vascular disease.

Thus, in certain embodiments, the methods and formulations of the invention deliver an amount of drug (e.g., dopamine agonist) in the TRNA therapy that would provide sub-therapeutic plasma levels if administered orally, but which is therapeutically effective when administered via TRNA therapy at the BONATH.

It is hypothesized by the inventor that a principal reason TRNA therapy is rapid in the onset of clinical effect (e.g., less than about 10-15 minutes with topical apomorphine) is that it operates through an “electro-chemical” process. Active drug compounded in an appropriate dermal penetration enhancing medium acts at free nerve endings, changing the neurochemistry of receptors at the neural synapse: apomorphine (dopamine and norepinephrine agonist), increasing dopamine and norepinephrine levels and improving neural transmission. After a point of receptor stimulation, neural (electrical) impulses are generated back to neuronal cell bodies residing in the spinal cord and brainstem: “afferent feed-back”. The nervous system functions through neurons generating electrical impulses and the release of neurochemicals/neuro-transmitters (serotonin, norepinephrine, dopamine, and acetylcholine, being the major ones) at neural receptor sites called “synaptic clefts”. Accordingly, the process in TRNA therapy may be considered analogous to an electrical capacitor discharging to perform a function, such as turning on a light switch. Viewed from this perspective, the rapid onset of clinical effect observed in TRNA therapy makes sense.

Alternatively, rapid as well as prolonged clinical effect may be achieved by a sustained-release dermal system employing the principles of TRNA therapy through patch application at the skin at the site of the injury. The location is critical in TRNA therapy, whereas, with a transdermal systemic patch, location is irrelevant.

Alternatively, TSD therapy, the traditional transdermal systemic delivery, operates on the principles of chemical gradients and fluid dynamics. These processes have associated inherent idiosyncrasies and variabilities; heart function as a pump for blood flow being one. Accordingly, despite the advantage of measurable drug levels, traditional transdermal systemic delivery involves a more circuitous route with slower clinical effect. This makes systemic transdermal patch delivery inappropriate for acute therapy.

The inventor has previously reported on the use of compounded topical apomorphine as a cream for the treatment of peripheral and spinal pain conditions. The proposed mechanism of action is based on the effect of apomorphine on nociceptive cutaneous nerve endings and spinal nerve roots which transmit pain impulses to the spinal cord dorsal horn and brain processing areas for interpretation as pain and other symptoms. Apomorphine possesses agonist (enhancing/stimulating) action on serotonin (5-HT), norepinephrine (NE), and dopamine (DA) receptors. 5-HT and NE are known to play a major role in the attenuation of pain signals from peripheral sites of injury which produce nociceptive (pain) input.

Endorphins and enkephalins produced by the body (endogenous opioids), or provided as opiate drugs and narcotics (exogenous opioids) also alleviate pain by inhibiting pain signals from sites of nerve injury. GABA, gamma butyric acid, a chemical produced by the body is also a pain inhibitor. On the other hand, substances that are excitatory to and enhance the pain process are: NMDA-glutamate, nitric oxide, and Substance P.

Studies indicate there are opioid receptors on free nerve endings of skin which may be affected by topically applied compounded opiate drugs to provide pain relief. The presence of opioid receptors and of endorphins in the central nervous system has been known since the 1970's. These play roles in pain processing and interpretation by the brain. However, with the knowledge these receptors also exist on skin nerve endings suggest they may be activated topically. Indeed, studies by Tennant indicate compounded morphine (in cold cream) when applied locally to the skin at areas of pain provided relief. Further, morphine levels were not detected in blood or urine, indicating lack of systemic absorption. It was concluded morphine acted directly on opiate receptors on nerve endings to produce pain relief and not systemically; that is, through the blood and then acting on the brain.

Topical Neuro-Affective Drug Therapy is the means by which a “neuro-active” drug (one acting to alter nerve function either “peripherally” in the extremities or spine; or “centrally,” in the brain, to treat symptoms attributable to the nervous system—pain, mood disorders, seizures, movement disorders such as Parkinson's disease or essential tremor, etc.) is applied to the skin as a compounded preparation to enhance penetration to the level of the free nerve-endings below the skin surface to exert therapeutic effects. Previously described has is the “nuchal” (back of the neck at the hairline, or “BONATH”) application of triptans (Migraderm using sumatriptan) for migraine headache, tizanidine (Tizoderm) for cervical spasm and tension headache; and, apomorphine for the Parkinson's disease symptoms of tremor, rigidity, speech difficulties, and other “non-motor” symptoms. Compounded morphine sulfate and more recently, tramadol have been applied at the nuchal region for the relief of both local peripheral and generalized pain with good results. The one time application doses used have been 2.5 mg for morphine sulfate and 40 mg for tramadol, respectively. Pain reduction in treated patients has been in the range of 30-60%. Over 20 patients have used these preparations on a regular basis with continued benefit. In fact, a number have been able to completely taper off their oral narcotics, using the nuchal delivery as their only method of pain therapy. Many systemic side-effects of narcotics such as lethargy, fatigue, cognitive slowing, and depression have significantly diminished in these patients, improving quality of life. Further, no significant narcotic withdrawal symptoms were noted.

When pain in an extremity or a specific region of the body is the result of an injury, nociceptive (pain-related nerve) input is provided through nerve-endings and small peripheral sensory nerves to the dorsal horn of the spinal cord and relayed to the brain for processing as pain and associated symptoms. Accordingly, these impulses may be modified or inhibited by drugs topically applied at strategic points along the course of nerve function: at point of injury, at strategic points of nerve branching in an extremity, at spinal nerve root entry zones, and at the nuchal region at the back of the neck. At the nuchal area, there occurs significant convergence of nerve input from the body to the CNS through trigeminal nerve, vagus nerve, and the sympathetic nerve afferent systems. At no other single point is there such a concentrated confluence of nerve impulses from the body to the central nervous system accessible for modification with topical drug application. The cutaneous nerve-endings at this location are part of the peripheral nerves which make up the cervical nerve roots that are joined by the trigeminal, vagal, and sympathetic nerves returning to the brain.

In the experience of the inventor, both generalized and isolated peripheral pain conditions may be treated by the nuchal application of the drugs described herein, e.g., morphine, apomorphine, and tramadol. However, where there is only a specific extremity or region of the body involved, it has been found that the pain relief to be superior when topical drug application is at several strategic sites along the nerve in addition to the nuchal area. For example, for treating severe foot pain at the instep from excessive running (tarsal tunnel syndrome and plantar fasciitis); to apply topical drug at the “tarsal tunnel” at the ankle, the lower back at the nerve root level supplying the foot, L5-S1; and, also at the nuchal region. In this way, pain impulses may be affected or blocked at numerous points, significantly reducing signals which reach the brain to be processed as pain. Depending on acuteness of the injury and lack of accompanying symptoms, localized treatment near the site of injury may be sufficient. However, when pain has been long-standing and there exist other complicating symptoms such as muscle spasms, fatigue, sleep disturbance, and alterations in mood; then, topical applications at other sites along the nerve supply as well as the nuchal region becomes necessary to capture the other neurally-activated aspects of a “chronic pain syndrome.”

In view of the presence of opioid receptors on cutaneous peripheral nerves and in the CNS, it is logical to topically administer opioids and opioid-like agonist drugs at strategic sites along the neural axis to optimize their pain-relieving function. The author has successfully used compounded morphine sulfate (MS) and tramadol topically to treat acute and chronic pain with applications at peripheral locations near the injury site/pain source as well as in conjunction with nuchal application. The dose for MS has been 2.5-5.0 mg and for tramadol, 40-80 mg, divided over two to three locations, as described herein.

Prior to using MS and tramadol, compounded apomorphine (1-2 mg doses) had been used peripherally and at spinal locations for both acute and chronic pain with success. This is previously reported. Apomorphine is known to act at NE, 5-HT, and dopamine (DA) receptors. Its effect on DA is used in the treatment of Parkinson's disease. Its effect on NE and 5-HT is likely responsible for is analgesic properties when administered topically as reported.

With activation of opioid receptors potentially providing improved pain relief, MS and tramadol were compounded for topical administration. Initial results suggest both these preparations provide significant pain relief within a relatively short period, 15-20 minutes which may last up to 4-6 hours. Reduction in pain has been reported in the 30-80% range. Significantly, at times relief was experienced by patients who were already on large amounts of systemic narcotics; either oral, by intra-thecal pump, or as transdermal patch. With topical delivery, a number of patients have been able to reduce or completely wean off systemic opioid drugs. This resulted in the reduction of the side-effects of lethargy, fatigue, and cognitive slowing commonly associated with systemic narcotic therapy. In these patients, the improvement in quality of life was significant. A program of drug rehabilitation has begun to wean patients off systemic narcotics, substituting pain management with topical neuro-affective therapy using MS and tramadol. Further, as a compounded topical preparation, these drugs are less amenable to modification for “recreational use” and abuse. Without the involvement of drug blood levels, the potential for addiction, tolerance, withdrawal, and of CNS depression with risk for over-dose are also diminished.

Morphine (MS Contin®, MSIR®, Avinza®, Kadian®, Oramorph®, Roxanol®, Kapanol®) is a potent opiate analgesic medication and is considered to be the prototypical opioid. Morphine is the most abundant alkaloid found in opium. As mentioned, the human body also produces small amounts of morphine-like substances and metabolizes them into a number of other active opiates. In 2003, there was discovery of endogenous morphine occurring naturally in the human body. There had been some thirty years of speculation on this subject as a receptor in human tissue that only reacted to morphine had previously been discovered: the mu3 opiate receptor.

In clinical medicine, morphine is regarded as the gold standard, or benchmark, of analgesics used to relieve severe or agonizing pain and suffering. Like other opioids, e.g. oxycodone (OxyContin®, Percocet® Percodan®), hydromorphone (Dilaudid®, Palladone®), and diacetylmorphine (heroin), morphine acts directly on the central nervous system (CNS) to relieve pain. As noted above, opioid receptors also exist on peripheral nerves. These, in turn, may be stimulated by the topical application of opioid drugs to provide pain relief by influencing opiate receptor neural impulse propagation to the brain.

Morphine has a high potential for addiction. Tolerance and psychological dependence develop rapidly, although physical addiction may take several months to develop. Diacetylmorphine (heroin) was synthesized from morphine in 1874 and brought to market by Bayer in 1898. Heroin is approximately 1.5-2 times more potent than morphine on a milligram-for-milligram basis. Due to the lipid solubility of diacetylmorphine, it is able to cross the blood-brain barrier faster than morphine, increasing the potential for addiction. Morphine became a controlled substance in the US under the Harrison Narcotics Tax Act of 1914, and possession without a prescription in the US is a criminal offense. Morphine was the most commonly abused narcotic analgesic in the world until heroin was synthesized and came into use. Even today, morphine is the most sought after prescription narcotic by heroin addicts when heroin is scarce, all other things being equal. The stop-gap drugs used by the largest absolute number of heroin addicts is probably codeine, with significant use also of dihydrocodeine, poppy straw derivatives like poppy pod and poppy seed tea, propoxyphene, and tramadol.

Tramadol hydrochloride (Ultram®, Tramal®, others) is a centrally acting opioid analgesic, used in treating moderate to severe pain. The drug has a wide range of applications, including treatment for restless leg syndrome and fibromyalgia. Tramadol possesses weak agonist actions at the μ-opioid receptor, releases serotonin, and inhibits the reuptake of norepinephrine.

Tramadol is a synthetic analog of the phenanthrene alkaloid codeine and, as such, is an opioid and also a prodrug (codeine is metabolized to morphine; tramadol is converted to O-desmethyltramadol). Opioids are chemical compounds which act upon one or more of the human opiate receptors. The euphoria and respiratory depression are mainly caused by the μ1 and μ2 receptors; the addictive nature of the drug is due to these effects as well as its serotonergic/noradrenergic effects. The opioid agonistic effect of tramadol and its major metabolite(s) are almost exclusively mediated by the substance's action at the μ-opioid receptor. This characteristic distinguishes tramadol from many other substances (including morphine) of the opioid drug class, which generally do not possess tramadol's degree of subtype selectivity.

Tramadol is used similarly to codeine, to treat moderate to moderately severe pain. Tramadol is somewhat pharmacologically similar to levorphanol (albeit with much lower μ-agonism), as both opioids are also NMDA-antagonists which also have SNRI activity. Tramadol is also molecularly similar to venlafaxine (Effexor) and has similar SNRI effects, with antinociceptive effects also observed. It has been suggested that tramadol could be effective for alleviating symptoms of depression, anxiety, and phobias because of its action on the noradrenergic and serotonergic systems, such as its “atypical” opioid activity. However, health professionals have not endorsed its use for these disorders, claiming it may be used only as a unique treatment.

The 2010 Physicians' Desk Reference (PDR) contains several warnings from the manufacturer, which were not present in prior years. The warnings include more compelling language regarding the addictive potential of tramadol, the possibility of difficulty breathing while on the medication, a new list of more serious side effects, and a notice that tramadol is not to be used in place of opiate medications for addicts. Tramadol is also not to be used in efforts to wean addict patients from opiate drugs, nor to be used to manage long-term opiate addiction.

Tramadol is usually marketed as the hydrochloride salt (tramadol hydrochloride); the tartrate is seen on rare occasions, and rarely, is available for both injection (intravenous and/or intramuscular) and oral administration. The most well known dosing unit is the 50 mg generic tablet made by several manufacturers. It is also commonly available in conjunction with APAP (Paracetamol, Acetaminophen) as Ultracet, in the form of a smaller dose of 37.5 mg tramadol and 325 mg of APAP. The solutions suitable for injection are used in patient-controlled analgesia pumps under some circumstances, either as the sole agent or along with another agent such as morphine.

Investigational uses for tramadol include diabetic neuropathy, postherpetic neuralgia, opiate withdrawal management/Anti-Depressant withdrawal aid (proven to be effective, especially with drawal from its distant relative Venlafaxine(Effexor)), obsessive-compulsive disorder, premature ejaculation.

Tramadol acts as a μ-opioid receptor agonist, serotonin releasing agent, norepinephrine reuptake inhibitor, NMDA receptor antagonist, 5-HT2C receptor antagonist, (α7)5 nicotinic acetylcholine receptor antagonist, TRPV1 receptor agonist, and M1 and M3 muscarinic acetylcholine receptor antagonist. The analgesic action of tramadol has yet to be fully understood, but it is believed to work through modulation of serotonin and norepinephrine in addition to its mild agonism of the μ-opioid receptor. The contribution of non-opioid activity is demonstrated by the fact that the analgesic effect of tramadol is not fully antagonised by the μ-opioid receptor antagonist naloxone. The serotonergic-modulating properties of tramadol give tramadol the potential to interact with other serotonergic agents. There is an increased risk of serotonin toxicity when tramadol is taken in combination with serotonin reuptake inhibitors (e.g., SSRIs). Tramadol is also thought to have some NMDA antagonistic effects, which has given it a potential application in neuropathic pain states.

Tramadol has inhibitory actions on the 5-HT2C receptor. Antagonism of 5-HT2C could be partially responsible for tramadol's reducing effect on depressive and obsessive-compulsive symptoms in patients with pain and co-morbid neurological illnesses. 5-HT2C blockade may also account for its lowering of the seizure threshold. However, the reduction of seizure threshold could be attributed to tramadol's putative inhibition of GABA-A receptors at high doses.

The overall analgesic profile of tramadol supports intermediate pain, especially chronic states. It is slightly less effective for acute pain than hydrocodone, but more effective than codeine. It has a dosage ceiling similar to codeine, a risk of seizures when overdosed, and a relatively long half-life making its potential for abuse relatively low.

Tramadol is associated with the development of physical dependence and a severe withdrawal syndrome. Tramadol causes typical opiate-like withdrawal symptoms as well as atypical withdrawal symptoms including seizures. The atypical withdrawal symptoms are probably related to tramadol's effect on serotonin and norepinephrine reuptake. Symptoms may include those of SSRI discontinuation syndrome, such as anxiety, depression, anguish, severe mood swings, aggressiveness, brain “zaps”, electric-shock-like sensations throughout the body, paresthesias, sweating, palpitations, restless legs syndrome, sneezing, insomnia, tremors, and headache among others. In most cases, tramadol withdrawal will set in 12-20 hours after the last dose, but this can vary. Tramadol withdrawal lasts longer than that of other opioids; seven days or more of acute withdrawal symptoms can occur as opposed to typically three or four days for other codeine analogues. It is recommended that patients physically dependent on pain killers take their medication regularly to prevent onset of withdrawal symptoms and this is particularly relevant to tramadol because of its SSRI and SNRI properties, and, when the time comes to discontinue their tramadol, to do so gradually over a period of time that will vary according to the individual patient and dose and length of time on the drug. It is apparent in community practice that dependence to this agent may occur after as little as three months of use at the maximum dose—generally depicted at 400 mg per day. However, this dependence liability is considered relatively low by health authorities. Tramadol is not currently scheduled by the U.S. DEA, unlike opioid analgesics. It is, however, scheduled in certain states. Nevertheless, the prescribing information for Ultram warns that tramadol “may induce psychological and physical dependence of the morphine-type”.

Due to the possibility of convulsions at high doses for some users, recreational use can be very dangerous. Tramadol can, however, via agonism of μopioid receptors, produce effects similar to those of other opioids, although not nearly as intense due to tramadol's much lower affinity for this receptor. Tramadol can cause a higher incidence of nausea, dizziness, loss of appetite compared with opiates which could deter abuse to some extent. Tramadol can help alleviate withdrawal symptoms from opiates, and it is much easier to lower the quantity of its usage, compared with opioids such as hydrocodone and oxycodone.

While useful in medical practice for acute pain related to injuries and post-surgical states and for the management of the more difficult chronic pain conditions, efforts are being made to curb their use—particularly, in the illicit arena. This is occurring at all levels of society, from state and federal governmental agencies to local grass-roots movements in communities across the country. The latter are initiated by families who have experienced the loss of loved ones to overdose and by law enforcement, whose services are strained by the crime associated with their sale and use. Even in situations where opiate drugs are “legally prescribed,” the question of their negative aspects out-weighing the positive is being continually raised. Initially prescribed for legitimate reasons, the long-term pharmacological effects of addiction and tolerance may subsequently involve the need for progressively higher doses. This begins to affect the user's quality of life through insidious decline in cognitive abilities with deficits in judgment, mood disorders of depression and anxiety, lack of motivation with employment issues, and disrupted relationships with divorce and other estrangements. Here, the cost is incalculable in monetary terms.

As significant as are the effects of physician prescribed chronic opioid use, the greater cost to society is in their abuse through “recreational use.” Here, the cost is paid by lost lives and by crime and violence related to their sale and use. It is obvious meaningful reform must take place soon. Some measures have already taken place. For instance, in the state of Florida, the prescribing of opiate drugs is being more strictly controlled. Long-term prescriptions are limited to “certified pain specialists,” while other M.D.'s may only prescribe a two-week's supply at a time. Further, there are restrictions on cash payment for these drugs. In Sarasota County (Florida), a one-year moratorium was recently enacted on the establishment of new pain clinics. There are increasing efforts at all levels to educate health care professionals on prescription drug misuse and to encourage registration for the Prescription Drug Monitoring Database. These are the beginning of more to come with respect to legislation and other measures to control drug abuse.

The unfortunate aspect of the above is they represent attempts at regulating and modifying altered human behavior that has occurred as the result of a predictable pharmacological phenomenon—addiction. Addictionologists (specialists who treat addiction) tell us a truly addicted person cannot help their addictive behavior and will resort to any means to satisfy their needs. The inability to refrain from continued self-destructive behavior is the essence of addiction.

The addiction from long-term opiate drug use occurs as the result of drug affecting receptors in areas of the brain which control pleasure, satiety, and memory—the amygdala, hippocampus, and other components of the limbic system which control human emotional response. Pain is the mechanism by which an organism is made aware there is injury or problem within the system which needs to be addressed. Initially, the pain may be primarily physical or emotional; but, after some time, it becomes a combination of both. In time, particularly with the use of opiate drugs, which mask or suppress pain perception without actually treating the offending pain causing problem, the physical and psychological aspects become entwined that it becomes impossible to separate the two. When this occurs “drug addiction” has taken place. In such situations, even when the pain causing entity is removed by surgery or other means, the person still “feels” the pain and need for medication.

The phenomenon of “neuro-plasticity” refers to the predictable manner in which the human brain functions when chronically exposed to chemical substances which affect the brain and which may be addictive. In simple terms, the brain in these situations “re-programs and re-sets” itself to require progressively higher amounts of opiate drug to satisfy the sense of need for pain relief. This has been termed pain “central hyper-sensitization” by neuro-physiologists with “central” referring to the central nervous system, the brain. The process could also be termed “brain-accommodated pain syndrome,” with addiction and associated components of emotional, behavioral, and personality changes impacting function. Early intense rehabilitative intervention may reverse effects. But, often, after protracted drug use and established dysfunctional behaviors, the brain's network circuitry may become programmed and “hard-wired” that there is little hope for recovery. In such situations, help may only exist in the form of the restricted use of less potent opiates with regular drug testing and strictly controlled prescriptions.

The majority of, if not all, patients who present with real physical pain for medical treatment never start with the intent of become chronically addicted to pain drugs despite its eventuality. Unfortunately, when it does occur, unless intense drug rehabilitation is pursued, they are destined to lives of despair and misery. From a medical and pharmacological perspective, what then is the solution? Pharmaceutical companies producing narcotics, under both federal and public pressure, are attempting to come up with means to prevent “reformulation” of their marketed products to discourage use in ways other than intended. However these measures still do not prevent the possibility of addiction and the other aspects of the central accommodation pain syndrome that occurs with their legal use. This is the consequence of systemic effects through their presence in the bloodstream affecting brain structures which leads to addiction.

The question then arises whether these potentially addictive drugs can be delivered or used in a manner to provide relief where bona fide pain exists but not cause addiction. Further, could the other “systemic” effects associated with these drugs also be minimized or prevented—those of respiratory suppression and cognitive slowing? Deaths that result from drug “over-dose” or poisoning noted by the Sarasota County Health Department are usually the result of cardio-respiratory arrest from combining opiates with benzodiazepines and alcohol. The cumulative interactions of these drugs in the body suppress vital brainstem functions, such as breathing and heart rate maintenance, resulting in death. Especially in inexperienced young people, the extent to which these combinations may be “safely” used is unclear, leading to tragic, unintentional over-dose.

To date, topical neuro-affective therapy has been successfully used as described herein with the triptan class of drugs (for migraine and tension headache), the muscle relaxant tizanidine (for muscle spasm states and spasticity) and with apomorphine (for tremor and other symptoms of Parkinson's disease, restless legs syndrome, and peripheral neuropathic pain). In all these applications, therapeutic benefit was achieved without the usual side-effects associated with these same drugs delivered in the traditional manner, by mouth or injection. With topical neuro-affective therapy, significantly lower doses may be used as compounded drug need only reach the free nerve-endings immediately below the skin surface. For example, to treat a migraine attack, the usual topical dose of sumatriptan (Imitrex) is 12.5 mg—as opposed to the 100 mg tablet oral dose. The time to clinical benefit is also much quicker with the triptan nuchal topical neuro-affective therapy: 10-15 minutes compared to close to an hour with the triptan tablet. In certain preferred embodiments of the present invention, the method of treatment comprises administration of the drug(s)

As mentioned, studies indicate there are opiate receptors on free nerve endings of skin which may be affected by topically applied compounded opiate drugs to provide pain relief. The presence of opiate receptors and of endorphins in the central nervous system has been known since the 1970's. These play roles in pain processing and interpretation by the brain. However, with the knowledge these receptors also exist on skin nerve endings suggest they may be activated topically. Indeed, studies by Tennant indicate compounded morphine (in cold cream) when applied locally to the skin at areas of pain provided relief. Further, morphine levels were not detected in blood or urine, indicating lack of systemic absorption. It was concluded morphine acted directly on opiate receptors on skin nerve endings to produce pain relief and not systemically—through blood and acting on the brain.

As “central hyper-sensitization” or “brain-accommodated pain syndrome” is the result of central pain processing involving brain opiate receptors with connections to their counterparts in the skin and other components of the peripheral nervous system, it would seem “central” pain phenomena could be modulated and influenced by drug action on corresponding receptors on skin free nerve-endings. This concept has held true in studies with the triptans and apomorphine using nuchal (back of the neck) topical neuro-affective therapy. Here, receptors and associated neural processes in the brain are activated by topical drug application to the skin at the back of the neck to bring about therapeutic benefit. It therefore proposed that compounded morphine and other opiates when topically administered to the nuchal region will have similar therapeutic effects as when systemically delivered but without undesirable side-effects. As mentioned, the skin at the nuchal region or back of the neck has particularly high concentration of nerve connections with the brain through the trigeminal, vagal, and sympathetic nerve systems. This provides significant feed-back to the brain from the skin nerve endings and nerves. Considering that the brain and skin are both formed from the same embryonic tissue, neuro-ectoderm, it makes sense these connections exist.

Accordingly, applied at low doses and as a cream rubbed onto the skin to affect the cutaneous (skin) nerve endings without absorption into the subcutaneous soft tissue and blood vessels, one may achieve pain relief from topical opiate drugs without the general systemic effects of addiction, tolerance (a pharmacological term for the need for progressively higher doses of drug for same effect), and CNS suppression. If these latter undesirable side-effects of opioid drugs could be minimized or prevented by topical neuro-affective therapy, their negative impact on society would also be curtailed. Opiates delivered in this manner could be prescribed for de nouveau pain patients to prevent addiction; and, to those already on chronic use, to conceivably reduce the negative effects of high dose chronic use by traditional methods.

Topical neuro-affective therapy, via both peripheral nerve activation and at the nuchal region provides an alternative method of using pain relieving drugs with significant potential harm in a safer way with diminished side-effects. This technology may be applied to the opioid class of potent narcotics such as morphine as well as to opiate receptor agonists like tramadol and the SNRI class of drugs which are also used to treat pain—venlafaxine (Effexor®), duloxetine (Cymbalta®), milnaciprin (Savella®), and the like.

One skilled in the art having the benefit of the information contained herein will appreciate that there are many classes of drugs which would be useful for topical peripheral de-afferentation therapy. These classes of drugs include, but are not limited to: dopamine agonists such as apomorphine; skeletal muscle relaxants such as tizanidine.

In certain embodiments, the drug is a dopamine agonist such as apomorphine (Apokyn®, APO-go®), pramipexole (Mirapexin®), ropinirole (Requip®), bromocriptine (Parlodel®), cabergoline (Cabaser®, Dostinex®), pergolide (Permax®, Celance®) rotigotine (Neupro®), mixtures of any of the foregoing, or other dopamine agonists known to those skilled in the art.

In certain embodiments, the drug is a dopamine agonist such as apomorphine (Apokyn®, APO-go®), pramipexole (Mirapexin®), ropinirole (Requip®), bromocriptine (Parlodel®), cabergoline (Cabaser®, Dostinex®), pergolide (Permax®, Celance®) rotigotine (Neupro®), mixtures of any of the foregoing, or other dopamine agonists known to those skilled in the art. One skilled in the art will appreciate that dopamine agonists other than apomorphine may be used in the formulations and methods of the present invention, and all such agents are meant to be encompassed by the term “dopamine agonists.” For example, such drugs include, but are not limited to, carbidopa (Sinemet®), dopamine agonists (Requip®, Rotigotine®, Mirapex®), COMT inhibitors (Entacapone®, Tocapone), rasagiline (Azilect®) (MAO inhibitors) and MAO-B inhibitors (Selegiline (Eldepryl®).

Skeletal muscle relaxants have played a significant role in alleviating stiffness, pain, and discomfort caused by muscle sprains, muscle spasms, spasticity, tension headache and tension-related migraines. Their mechanism of action can be attributed to their direct effect on skeletal muscles (e.g., direct acting skeletal muscle relaxants such as dantrolene) or their ability to reduce spasticity by increasing pre-synaptic inhibition of motor neurons, inhibiting monosynaptic or polysynaptic reflexes at the spinal level (e.g., centrally acting skeletal muscle relaxants such as tizanindine and baclofen).

In certain embodiments, the active agent(s) is a skeletal muscle relaxant. The skeletal muscle relaxants for use in the present invention include centrally acting skeletal muscle relaxants, direct acting skeletal muscle relaxants and any combinations or mixtures thereof.

Centrally acting skeletal muscle relaxants include, but are not limited to for example and without limitation, afloqulone, baclofen, botulin toxins, carisoprodol, chlormezanone, chlorphenesin carbamate, chlorzoxazone, cyclobenzaprine, clonazepam, diazepam, eperisone, idrocilamide, inaperisone, mephenesin, mephenoxalone, methocarbamol, metaxalone, mivacurium chloride, orphenadrine, phenprobamate, pridinol mesylate, quinine, tetrazepam, thiocolchicoside, tizanidine, tolperisone, pharmaceutically acceptable salts thereof, active metabolites thereof, prodrugs thereof and mixtures thereof. Preferably the skeletal muscle relaxant is tizanidine base, tizanidine hydrochloride or any pharmaceutically acceptable salts thereof, prodrugs thereof or mixtures thereof.

Direct acting skeletal muscle relaxants include dantrolene.

Tizanidine is a centrally acting α2-adrenergic agonist. Tizanidine possesses an imidizole structure similar to that of clonidine (anti-hypertensive) and other α2-adrenergic agonists. Tizanidine is completely absorbed after oral administration with its peak effect occurring within about 1 to about 2 hours. The mechanism of action of tizanidine is related to its presumed ability to increase presynaptic inhibition of motor neurons thereby reducing spasticity with its greatest effect asserted on polysynaptic pathways.

In certain preferred embodiments, the formulations of the present invention contain a skeletal muscle relaxant base, pharmaceutically acceptable salt thereof, active metabolite thereof, or pro-drug thereof (e.g., tizanidine hydrochloride) as the skeletal muscle relaxant. When the skeletal muscle relaxant is tizanidine or pharmaceutically acceptable salt thereof, active metabolite thereof, or prodrug thereof, the amount of tizanidine present in the formulation is in a range from about 0.25 mg to about 2 mg, and preferably from about 0.4 mg to about 0.8 mg. In certain other preferred embodiments the amount of tizanidine included in a topical unit dose formulation is from about 0.2 mg to about 4 mg.

For comparative purposes, prior art topical doses of skeletal muscle relaxants range from about 10 mg to about 50 mg which is more than 10 to 100 times greater than the dosage range for the skeletal muscle relaxants of the present invention.

In addition, oral doses of certain skeletal muscle relaxants are as follows: carisoprodol 350 mg; chlorphenesin 400 mg; chlorzoxazone 250 mg; cyclobenzaprine 10 mg; metaxalone 800 mg; methocarbamol 1 gm to 1.5 gm; tizanidine 4 mg; orphenadrine 100 mg; diazepam 2 mg to 10 mg; baclofen 5 mg to 20 mg; and dantrolene 25 mg to 100 mg. Therefore, one skilled in the art can readily determine therapeutically equivalent doses of skeletal muscle relaxants that may be useful in the present invention. However, it is noted that the differences in oral doses may not directly correspond to the differences in doses that are therapeutically effective via transdermal delivery of the skeletal muscle relaxant. Factors such as metabolism of the skeletal muscle relaxant, the ability of the drug to pass through the skin, among others, may affect the amount of skeletal muscle relaxant necessary to provide a therapeutic effect. One skilled in the art would readily understand this and adjust for the same.

In certain embodiments, the active agent(s) is an opioid analgesic (opioid agonist), administered to the area(s) in an effective amount to provide the desired analgesic effect. Opioids, also known as opioid agonists, are a group of drugs that exhibit opium or morphine-like properties. The opioids are employed primarily as moderate to strong analgesics, but have many other pharmacological effects as well, including drowsiness, respiratory depression, changes in mood and mental clouding without a resulting loss of consciousness. Opioids act as agonists, interacting with stereospecific and saturable binding sites in the brain and other tissues. Endogenous opioid-like peptides are present particularly in areas of the central nervous system that are presumed to be related to the perception of pain; to movement, mood and behavior, and to the regulation of neuroendocrinological functions. Opium contains more than twenty distinct alkaloids. Morphine, codeine and papaverine are included in this group.

Opioid analgesics which are useful in the present invention include all opioid agonists or mixed agonist-antagonists, partial agonists, including but not limited to alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tramadol, mixtures of any of the foregoing, salts of any of the foregoing, and the like., the term “opioid” for the purposes of the present invention is considered to encompass synthetic entities with morphine-like actions, and all exogenous substances that bind stereo-specifically to any of several subspecies of opioid receptors and produce agonist actions.

In certain preferred embodiments, the opioid agonist or analgesic is selected from the group consisting of tramadol, hydrocodone, morphine, hydromorphone, oxycodone, codeine, levorphanol, meperidine, methadone, or salts thereof, or mixtures thereof. In certain preferred embodiments, the opioid agonist is hydrocodone. Equianalgesic doses of these opioids, in comparison to a 15 mg dose of hydrocodone, are set forth in Table 1 below:

TABLE 1
Equianalgesic Doses of Opioids

| Opioid | Calculated Dose (mg) |
|---|---|
| Oxycodone | 13.5 |
| Codeine | 90.0 |
| Hydrocodone | 15.0 |
| Hydromorphone | 3.375 |
| Levorphanol | 1.8 |
| Meperidine | 135.0 |
| Methadone | 9.0 |
| Morphine | 27.0 |

The invention disclosed herein is meant to encompass all pharmaceutically acceptable salts thereof of the disclosed opioid agonists. Some of the opioid agonists encompassed within the present invention may contain one or more asymmetric centers and may thus give rise to enantiomers, diastereomers, and other stereoisomeric forms. The present invention is also meant to encompass the use of all such possible forms as well as their racemic and resolved forms and mixtures thereof. When such compounds contain olefinic double bonds or other centers of geometric asymmetry, and unless specified otherwise, it is intended to include both E and Z geometric isomers. All tautomers are intended to be encompassed by the present invention as well. As used herein, the term “stereoisomers” is a general term for all isomers of individual molecules that differ only in the orientation of their atoms is space. It includes enantiomers and isomers of compounds with more than one chiral center that are not minor images of one another (diastereomers). The term “chiral center” refers to a carbon atom to which four different groups are attached. The term “enantiomer” or “enantiomeric” refers to a molecule that is nonsuperimposeable on its minor image and hence optically active wherein the enantiomer rotates the plane of polarized light in one direction and its minor image rotates the plane of polarized light in the opposite direction. The term “racemic” refers to a mixture of equal parts of enantiomers and which is optically inactive. The term “resolution” refers to the separation or concentration or depletion of one of the two enantiomeric forms of a molecule.

In certain preferred embodiments, the opioid analgesic is selected from tramadol, morphine, oxycodone, oxymorphone, hydromorphone, hydrocodone, pharmaceutically acceptable salts thereof, and mixtures thereof. In certain preferred embodiments, the opioid analgesic is a non-narcotic opioid such as tramadol. In certain preferred embodiments, tramadol is applied topically as the hydrochloride salt in an amount from about 20 to about 40 mg. In certain preferred embodiments, morphine is applied topically as its sulfate salt in an amount from about 2.5 mg to about 5 mg. In other preferred embodiments a narcotic or non-narcotic opioid analgesic is used in accordance with the present invention in a equianalgesic dose, e.g., as known to those skilled in the art and/or as set forth in Table 1 above.

In certain embodiments, the drug used in the methods of the present invention is an SNRI (serotonin-norepinephrine reuptake inhibitor). SNRIs are used in the treatment of major depression and other mood disorders, anxiety disorders, obsessive-compulsive disorder (OCD), attention deficit hyperactivity disorder (ADHD), chronic neuropathic pain, fibromyalgia syndrome (FMS), and for the relief of menopausal symptoms. SNRIs act upon and increase the levels of two neurotransmitters in the brain, i.e., serotonin and norepinephrine. This can be contrasted with selective serotonin reuptake inhibitors (SSRIs), which only act on serotonin. Non-limiting examples of SNRIs which may be used in the methods of the present invention include venlafaxine, desvenlafaxine, duloxetine, milnacipran, levomilnacipran, sibutramine, bicifadine, SEP-227162, and LY 2216684. Generally, it is contemplated that the unit dose initially applied at a location(s) of the SNRI will be about one-half of the starting oral dose of that drug. For example, that would translate to an initial topical dose of about 37.5 mg for venlafaxine, an initial topical dose of about 15 mg for duloxetine, and an initial topical dose of about 7.5 mg for milnacipran.

In certain embodiments, the formulations described herein are fast acting. For example, the symptoms associated with migraine and/or cluster headache, muscle sprain, muscle spasm, spasticity, tension related headache, tension related migraine and related conditions associated with muscle tension and pain are relieved within about 2 hours, preferably within about 5 minutes to about 2 hours, within about 5 minutes to about 1 hour and most preferably within about 5 minutes to about 30 minutes after application of the formulation. In certain preferred embodiments, the formulations of the present invention provide relief from migraine and/or cluster headache, muscle sprain, muscle spasm, spasticity, tension related headache, tension related migraine and related conditions associated with muscle tension and pain within from less than 1 minute to about 2 hours, from about 1 minute to about 2 hours, and most preferably from about 1 minute to about 15 minutes.

The methods of the present invention may also, if desired, involve pre-treatment of the skin with an enhancer to increase the permeability of the skin to the applied drug. The methods of the present invention may include pre-treatment or “prepping” of the skin area with a substance that opens up the skin pores. Additionally, the methods of the present invention may include, if desired, pre-treatment or “prepping” of the skin with an alcohol swab or the like to rid the area of dirt, make-up, oil, and the like, prior to application of the drug.

### Formulations

All currently approved therapies for the conditions described above reach the central nervous system through the systemic circulation. Cerebral blood flow to brainstem structures is through the posterior circulation, via the vertebral and basilar arteries and their branches. In view of the undesirable side-effects associated with this form of drug delivery to the brain, it makes sense that targeted regional delivery to the brainstem is sought. Topical delivery of currently used drugs compounded in an appropriate “dermal penetration enhancer” and applied in cream/gel form or as a sustained-release patch at the posterior cervical region (back of the neck) at the hairline is such a method. Lipoderm® is an example of an effective commercially available compounding medium. However, one skilled in the art will recognize that topical carriers meeting the specific chemical requirements of an individual drug can be formulated for maximum efficiency in topical delivery.

The formulations of the present invention are prepared such that the drug(s) may be delivered acutely as single dose applications as cream/gel/ointment or as a sustained release topical patch, depending on the condition treated and associated symptom complex in the individual patient. The critical point, again, is in the location of the application: at the back of neck at the hair-line for access to posterior cervical afferents with free nerve endings under the surface of the skin. Through feedback connections with vagal and trigeminal afferent systems, this results in ultimate effect on brainstem structures.

By virtue of the method of treatment described herein, the disease state/condition to be treated may be treated much faster and more effectively than such prior art modes of administration.

In certain embodiments of the present invention, the method of treating a human patient comprises applying a topical formulation which comprises a drug suitable for topical administration, which is useful for the treatment of a disease state or condition treatable via the topical brainstem afferent stimulation (de-afferentation) drug therapy described herein.

The methods of the present invention may also, if desired, involve pre-treatment of the skin with an enhancer to increase the permeability of the skin to the applied drug. The methods of the present invention may include pre-treatment or “prepping” of the skin area with a substance that opens up the skin pores. Additionally, the methods of the present invention may include, if desired, pre-treatment or “prepping” of the skin with an alcohol swab or the like to rid the area of dirt, make-up, oil, and the like, prior to application of the drug.

In certain embodiments, the topical formulation of the present invention comprises a drug in an amount which is therapeutically effective when administered topically at the at the back of neck at the hair-line for access to posterior cervical afferents with free nerve endings under the surface of the skin, but which provides a plasma concentration which is subtherapeutic if orally administered.

In certain embodiments, by applying the formulation of the present invention comprising a dose of drug at the back of neck at the hair-line for access to posterior cervical afferents with free nerve endings under the surface of the skin, it may be possible for the use of lower doses of drug or faster relief of the headache than if applied to the trunk or limbs of a human patient, and the lower plasma levels of drug which result from lower doses may thereby reduce unwanted side effects of the drug.

The topical formulations of the present invention (e.g., ointment, gel, cream, or the like), must be suitable for topical administration of a drug, i.e., must contain pharmaceutically acceptable excipients compatible with application to the skin tissue, and may optionally contain a sufficient amount of an enhancer composition as described hereinafter.

In certain embodiments, in addition to the drug (e.g., dopamine agonist), the topical formulations and/or transdermal therapeutic systems of the present invention may include at least one adjuvant such as a penetration enhancer, anti-oxidant, stabilizer, carrier, or vehicle. Additionally or alternatively, the present invention may include the application of electric current (iontophoresis) for enhancing permeation of the dopamine agonist.

In certain embodiments, the topical formulations comprising a drug in an ointment, gel, cream or the like, will typically contain on the order of about 0.001 to about 80% by weight, preferably 0.01 wt. % to 50 wt. % drug, and about 0 wt. % to about 50.0 wt. %, preferably from about 1 wt. % to about 30 wt. % of a permeation enhancer composition, with the remainder of the composition comprising a carrier or vehicle. In certain preferred embodiments, the drug is included in a cream or gel or ointment in a concentration of, e.g., 1 mg drug/ml of carrier (e.g., Lipoderm). However, it is to be understood that one skilled in the art can increase the amount of carrier or change the carrier and maintain or improve efficacy of the topical formulation for TRNA therapy.

In certain embodiments, the topical formulations comprising a dopamine agonist in an ointment, gel, cream or the like, will typically contain on the order of about 0.001 to about 80% by weight, preferably 0.01 wt. % to 50 wt. % dopamine agonist, and about 0 wt. % to about 50.0 wt. %, preferably from about 1 wt. % to about 30 wt. % of a permeation enhancer composition, with the remainder of the composition comprising a carrier or vehicle. In certain preferred embodiments, the dopamine agonist is apomorphine and is included in a cream or gel or ointment in a concentration of, e.g., 1 mg drug/ml of carrier (e.g., Lipoderm). However, it is to be understood that one skilled in the art can increase the amount of carrier or change the carrier and maintain or improve efficacy of the topical formulation for TRNA therapy. In certain preferred embodiments, the drug is applied as a unit dose at the BONATH in immediate release form (e.g., cream, ointment or gel) for acute treatment with a dopamine agonist as would be beneficial to a person suffering from, e.g., Parkinson's disease or impotence/male erectile dysfunction. In such instances, it is preferred that the concentration of dopamine agonist included in the unit dose is from about 0.25 mg to about 4 mg, based on apomorphine, or an therapeutically equivalent amount of another dopamine agonist as described herein.

Suitable permeation enhancers may also be included in the formulations. Such enhancers include, but are not limited to, dimethylsulfoxide (DMSO), N,N-dimethylacetamide (DMA), decylmethylsulfoxide (C10 MSO), polyethylene glycol monolaurate (PEGML), propylene glycol (PG), PGML, glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond, Va.), alcohols, and the like. The permeation enhancer may also be a vegetable oil as described in U.S. Pat. No. 5,229,130 to Sharma. Such oils include, for example, safflower oil, cotton seed oil and corn oil.

Additional enhancers for use in conjunction with the present invention are lipophilic compounds having the formula [RCOO]n R′, wherein n is 1 or 2 and R is C1-C16 alkyl optionally substituted with 1 or 2 hydroxyl groups, and R′ is hydrogen or C1-C16 alkyl optionally substituted with 1 or 2 hydroxyl groups. Within this group, a first subset of compounds are represented by the formula [CH3(CH2)m COO]n R′ in which m is an integer in the range of 8 to 16, n is 1 or 2, and R′ is a lower alkyl (C1-C3) residue that is either unsubstituted or substituted with one or two hydroxyl groups. Preferred enhancers within this group include an ester which is a lower alkyl (C1-C3) laurate (i.e., m is 10 and n is 1) such as “PGML”. It will be appreciated by those skilled in the art that the commercially available material sold as “PGML” is typically although not necessarily a mixture of propylene glycol monolaurate itself, propylene glycol dilaurate, and either propylene glycol, methyl laurate, or both. Thus, the terms “PGML” or “propylene glycol monolaurate” as used herein are intended to encompass both the pure compound as well as the mixture that is typically obtained commercially. Also within this group is a second subset of compounds, namely, esters of fatty alcohols represented by the formula CH3(CH2)m-O—CO—CHR1R2, in which R1 and R2 are independently hydrogen, hydroxyl, or lower alkyl (C1-C3), and m is as above. Particularly preferred enhancers within this group are lauryl lactate and myristyl lactate. In addition, a third subset of compounds within this group are analogous fatty acids, i.e., acids having the structural formula CH3(CH2)m COOH where m is as above. A particularly preferred acid is lauric acid.

Other enhancer compositions are wherein a lipophilic compound as just described, particularly PGML is combined with a hydrophilic compound, such as a C2-C6 alkanediol. One preferred hydrophilic enhancer within this group is 1,3-butanediol. Such enhancer compositions are described in detail in PCT Publication No. WO 95/05137, published Feb. 23, 1995, herein incorporated by reference. Another hydrophilic enhancer that may be included in these compositions is an ether selected from the group consisting of diethylene glycol monoethyl ether (Transcutol) and diethylene glycol monomethyl ether. Such enhancer compositions are described in detail in U.S. Pat. Nos. 5,053,227 and 5,059,426 to Chiang et al., the disclosures of which are herein incorporated by reference.

Other enhancer compositions may include mixture or combinations of any of the aforementioned enhancers, and the like.

In certain embodiments the topical formulation may include at least one water-insoluble, pharmacologically approved, alkyl cellulose or hydroxyalkyl cellulose, and the like. Alkyl cellulose or hydroxyalkyl cellulose polymers for use in this invention include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross linking agent may be used to modify the polymer's characteristics. For example, esters such as dibutyl or diethyl phthalate, amides such as diethyldiphenyl urea, vegetable oils, fatty acids and alcohols such as acid oleic and myristyl may be used in combination with the cellulose derivative.

In certain embodiments, the topical formulation may further include hydrocarbons such as liquid paraffin, vaseline, solid paraffin, microcrystalline wax, etc.; higher aliphatic alcohols such as cetyl alcohol, hexadecyl, alcohol, stearyl alcohol, oleyl alcohol, etc.; esters of higher fatty acids with higher alcohols such as beeswax, etc.; esters of higher fatty acids with lower alcohols such as isopropyl myristate, isopropyl palmitate, etc.; vegetable oils, modified vegetable oils, hydrous lanolin and its derivative, squalene, squalane; higher fatty acids such as palmitic acid, stearic acid, etc. and the like.

In certain embodiments, the topical formulation may further include emulsifiers and dispersing agents which include, for example, anionic, cationic and nonionic surfactants. Nonionic surfactants are preferred because of their low levels of irritation to skin. Typical of nonionic surfactants are fatty acid monoglycerides such as glyceryl monostearate, etc.; sorbitan fatty acid esters such as sorbitan monolaurate, etc.; sucrose fatty acid esters; polyoxyethylene fatty acid esters such as polyoxyethylene stearate, etc.; and polyoxyethylene higher alcohol ethers such as polyoxyethylene cetyl ether, polyoxyethylene oleyl ether, etc.

In certain preferred embodiments, the topical TRNA formulation is aqueous-based.

In certain embodiments of the present invention, the topical formulation may include a gelling agent such as methylcellulose, ethylcellulose, hydroxyethylcellulose, hydroxypropyl-cellulose, hydroxypropylmethylcellulose, carboxymethylcellulose, carbomer, and the like. Examples of pharmaceutical compositions which rely upon an aqueous gel composition as a vehicle for the application of a drug are U.S. Pat. Nos. 4,883,660; 4,767,619; 4,511,563; 4,861,760; and 5,318,780, the disclosures of which are herein incorporated by reference.

The topical formulation may further include one or more preservatives, stabilizers, or anti-oxidants.

Examples of preservatives that may be used in a formulation according to the present invention include, but are not limited to, bacteriostatic compounds and other preservatives suitable for topical administration including various alcohols, sorbic acid and salts and derivatives thereof, ethylenediamine, monothioglycerol, and thimerosal.

Examples of stabilizers that may be present in a formulation according to the present invention include pH buffers suitable for topical administration, complexing agents, chelating agents and the like.

Examples of anti-oxidants that may be used in a formulation according to the present invention include ascorbic acid and its derivatives, e.g., ascorbyl palmitate, as well as butylated hydroxyanisole, butylated hydroxytoluene, sodium bisulfite, sodium metabisulfite, and others.

Other adjuvants that may be included in the drug formulation include carriers, tackifiers, pigments, dyes, and other additives that do not adversely affect the mechanical or adhesive properties of the formulation.

“Carriers” or “vehicles” as used herein refer to carrier materials suitable for transdermal drug administration, and include any such materials known in the art, e.g., any liquid, gel, emulsion, solvent, liquid diluent, solubilizer, or the like, which is nontoxic and which does not interact with other components of the composition in a deleterious manner. The term “carrier” or “vehicle” as used herein may also refer to stabilizers, crystallization inhibitors, dispersing agents or other types of additives useful for facilitating transdermal drug delivery. It will be appreciated that compounds classified as “vehicles” or “carriers” may sometimes act as permeation enhancers, and vice versa, and, accordingly, these two classes of chemical compounds or compositions may sometimes overlap.

Carrier materials suitable for use in the instant compositions include those well-known for use in the cosmetic and medical arts as bases for ointments, lotions, salves, aerosols, suppositories and the like. Suitable carriers include, for example, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers herein include for example alcohols, including both monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

In certain preferred embodiments of the present invention where it is desired that the drug (.e.g., dopamine agonist) is administered chronically, the formulations of the present invention may be formulated as a transdermal delivery system (also referred to herein as a transdermal therapeutic system) such as a transdermal patch, a transdermal plaster, a transdermal disc, iontophoretic transdermal device, or the like. Such formulations are recognized by those skilled in the art as providing a release of drug and absorption into the skin of the patient in a sustained manner over an extended period of time (e.g., 1-7 days). In such embodiments of the present invention, the transdermal delivery system comprises, e.g., a dopamine agonist contained in a reservoir or a matrix, and an adhesive which allows the transdermal patch to adhere to the skin, allowing the passage of the active agent from the transdermal patch through the skin of the patient. In preferred embodiments, the transdermal patch is applied topically at the back of the neck at the hairline (“BONATH”) so as to achieve topical regional neuro-affective therapy (“TRNA THERAPY”) as described herein. In embodiments in which the drug is contained in a transdermal patch, it is contemplated that the drug will be absorbed more slowly and the transdermal patch will provide a sustained release and prolonged therapeutic effect, as compared, e.g., to a cream or ointment intended to provide an immediate release of the drug and rapid onset of the TRNA therapy.

In certain embodiments, the transdermal delivery devices, as well as other transdermal delivery systems in accordance with the invention can be made in the form of an article such as a tape, a patch, a sheet, a dressing or any other form known to those skilled in the art. Generally the device will be in the form of a patch of a size suitable to deliver a unit dose of serotonin agonist through the skin. The drug may be introduced into a transdermal therapeutic system in different forms (solid, in solution, in dispersion); it may also be microencapsulated.

In certain embodiments the present invention provides a transdermal therapeutic system comprising a drug (e.g., serotonin agonist) in an amount that would provide sub-therapeutic plasma levels if administered orally, but is therapeutically effective when administered via transdermal delivery at the headache region.

A transdermal delivery system for use in accordance with the present invention can also be constructed with an enhancer composition and other ingredients described hereinabove with respect to the topical formulation. Preferably, the transdermal delivery system is formulated for the prolonged delivery of a drug (e.g., dopamine agonist) as would be beneficial to a person suffering from, e.g., Parkinson's disease or impotence/male erectile dysfunction. The targeted skin flux for delivery of a particular drug can be achieved by adjusting vehicle composition and vehicle loading, as well as by adjusting the surface area through which the compositions are administered to skin.

In certain preferred embodiments, the transdermal delivery system (e.g., patch) is formulated to deliver from about 4 mg to about 50 mg of the dopamine agonist per each 24 hours through the skin of the patient, based on apomorphine, or a therapeutically equivalent amount of a suitable alternative dopamine agonist as described herein. In embodiments in which the transdermal delivery system is intended to be applied to the skin at the BONATH for multiple days, the transdermal delivery system (e.g., patch) is formulated to provide a flux rate over the useful life of the system such that a similar amount (e.g., mean dose) is delivered on a daily basis until the system is removed and replaced with a fresh system.

The transdermal delivery system used in the present invention may be prepared, for example, in accordance with U.S. Pat. Nos. 5,069,909; 4,806,341; 5,026,556; 4,588,580; 5,016,652; 3,598,122; 4,144,317; 4,201,211; 4,262,003; and 4,379,454; all of which are incorporated herein by reference.

Additionally, the transdermal delivery system used in the present invention may be in accordance with U.S. Pat. No. 6,689,379, hereby incorporated by reference, which system is a matrix or reservoir system which comprises: at least one pharmaceutical active agent selected from the group consisting of basic pharmaceutical active agents and neutral pharmaceutical active agents (such as rivastigmine); and a pressure-sensitive adhesive comprising a polyacrylate polymer, wherein said polyacrylate polymer has a polyacrylate backbone containing monomer units selected from the group consisting of acrylic acid, methacrylic acid and ester derivatives of acrylic or methacrylic acid, and said monomer units comprise at least 50% (w/w) relative to a mean polymer mass of said polyacrylate polymer, a total amount of monomers selected from the group consisting of non-esterified acrylic acid and non-esterified methacrylic acid is 0.5 to 10.0% (w/w) relative to the mean polymer mass of said polyacrylate polymer, and the carboxyl groups of said non-esterified acrylic and methacrylic acid monomers are present stoichiometrically at 5 to 100% in the form of alkali salts or alkaline-earth salts, said salts being reaction products of a neutralization reaction of an alcoholic solution of an alkaline hydroxide or an alkaline-earth hydroxide with said acrylate polymer(s), or of a neutralization reaction of an alkali alcoholate or an alkaline-earth alcoholate with said acrylate polymer(s).

In certain embodiments, the dosage form can be a transdermal patch comprising a laminated composite for administering the drug (e.g., dopamine agonist) to an individual transdermally comprising: (a) a polymer backing layer that is substantially impermeable to the dopamine agonist; and (b) a reservoir layer comprising a water-base acrylate pressure-sensitive adhesive, 1 to 12% by weight serotonin agonist and 2 to 25% by weight of a permeation enhancer comprising propylene glycol monolaurate in combination with capric acid or oleic acid, wherein the skin contact area of the composite is 10 to 100 cm2.

The dosage form can be a transdermal patch comprising (a) a polar solvent material selected from the group consisting of C3-C4 diols, C3-C6 triols, and mixtures thereof; and (b) a polar lipid material selected from the group consisting of fatty alcohol esters, fatty acid esters, and mixtures thereof; wherein said polar solvent material and said polar lipid material are present in a weight ratio of solvent material:lipid material of from about 60:40 to about 99:1.

In certain embodiments, the dosage form also comprises a transdermal plaster comprising: (1) a film layer which comprises a polyester film of 0.5 to 4.9 microns thickness, 8 to 85 g/mm strength, respectively in the two directions intersecting substantially at right angles, 30 to 150% elongation, in the two directions intersecting substantially at right angles and an elongation ratio of A to B of 1.0 to 5.0, wherein A and B represent data in two directions intersecting at right angles, and A is greater than B, and wherein said polyester film comprises 0.01 to 1.0% by weight, based on the total weight of said polyester film, of solid fine particles in which (a) the average particle size is 0.001 to 3.0 microns, and (b) the average particle size is substantially not more than 1.5 times the thickness of said polyester film; and (2) an adhesive layer (a) which is composed of an adhesive containing said serotonin agonist and further wherein said adhesive layer (a) is laminated on said film layer over the surface in a 2 to 60 microns thickness.

In certain embodiments, the dosage form can be a transdermal disc comprising: (a) a backing layer which is substantially impervious to the dopamine agonist; and (b) a polymer matrix disc layer which is adhered to said backing layer and which has microdispersed therein said serotonin agonist, said polymer being bioacceptable and permitting said serotonin agonist to be transmitted for transdermal absorption, the dopamine agonist being stable in said polymer matrix.

In certain embodiments, the topical formulation or transdermal therapeutic system may further comprise another active ingredient in combination with the first drug (e.g., dopamine agonist), e.g., analgesics, antimimetics, psychopharmacologic agents, or sedatives.

The present invention is contemplated to encompass all transdermal formulations, e.g., the technologies described above, with the inclusion of a drug (e.g., dopamine agonist(s)), such that the administration of a drug useful for treatment of disease state or condition in humans via topical brainstem afferent stimulation (de-afferentation) therapy via topical administration. Therefore, modifications of the invention via, e.g., the choice and/or amount of drug are considered to be obvious variations of this disclosure and within the scope of the appended claims.

The present invention also contemplates the administration of the drug (e.g., dopamine agonist) directly below the skin to affect direct brainstem afferent stimulation to the free nerve endings under the epidermis. Such administration may be effected as an injection (e.g., subcutaneous injection) or implantation of the drug in immediate release or sustained release form. It will be appreciated by those skilled in the art that providing the drug in sustained release form and administering it in a suitable form below the skin may provide benefits, including less frequent administration (e.g., in chronic therapy).

In certain embodiments of the invention, the drug (e.g., dopamine agonist) can be formulated for controlled or sustained delivery at the BONATH via incorporation into a biocompatible and implantable polymer which can be in the form of microparticles or an implantable insert, or a liquid that forms a gel or colloid or a semi-solid after injection (thereby encapsulating the drug and allowing it to be released in a prolonged and controlled manner at the desired site). For chronic conditions (e.g., Parkinson's) or desired prolonged effect, it is contemplated that a drug depot or reservoir may be created under the skin at the BONATH, which then provides a sustained release of the drug in proximity to the desired nerve endings and which may be replenished or replaced at the end of the dosing interval. It is contemplated that such administrations of the drug (e.g., dopamine agonist) may provide a prolonged therapeutic effect for at least about 3 days, preferably at least about 7 days, or longer. Such formulations may be administered in certain embodiments as, for example, a subcutaneous depot.

Implants are placed subcutaneously by making an incision in the skin and forcing the implants between the skin and the muscle. At the end of their use, if not dissolved, these implants are surgically removed. U.S. Pat. No. 4,244,949, hereby incorporated by reference, describes an implant which has an outer matrix of an inert plastic such as polytetrafluoroethylene resin. Examples of this type of implantable therapeutic system are Progestasert IUD and Ocusert system. It is contemplated that such systems can be appropriately modified by one skilled in the art for use in conjunction with the present invention. A commercially available product, Norplant®, which is an implant having a core containing levonorgestrel as the active substance, and where the core it surrounded by a membrane of a silicone elastomer of poly(dimethylsiloxane) (PDMS). Another preparation of this kind is Jadelie®, in which the core is a poly(dimethylsiloxane) based matrix with levonorgestrel dispersed therein. The membrane is an elastomer made from PDMS and silica filler, which, besides giving necessary strength properties to the membrane, also retards the permeation of the active agent through the membrane. U.S. Pat. No. 3,854,480, hereby incorporated by reference, describes a drug delivery device, e.g. an implant, for releasing a drug at a controlled rate for a prolonged period of time. The device has a core of a matrix in which the drug is dispersed. The core is surrounded by a membrane that is insoluble in body fluids. The core matrix as well as the membrane are permeable to the drug by diffusion. The materials of the core and the membrane are chosen so that the drug diffuses through the membrane at a lesser rate than through the core matrix. Thus, the membrane controls the release rate of the drug. As a suitable polymer for the core matrix is mentioned poly(dimethylsiloxane) (PDMS), and as suitable polymers for the membrane are mentioned polyethylene and a copolymer of ethylene and vinyl acetate (EVA). It is contemplated that the above systems may be adapted by one skilled in the art to deliver the drug (e.g., dopamine agonists) in accordance with the present invention.

One device which may be adapted by one skilled in the art for use in the present invention is described in U.S. Pat. No. 5,968,542 (Tipton), hereby incorporated by reference, which describes a high viscosity liquid controlled delivery system as a medical or surgical device is provided that includes: (i) a non-polymeric, non-water soluble liquid carrier material (HVLCM) of viscosity of at least 5,000 cP at 37.degree. C. that does not crystallize neat under ambient or physiological conditions; and, optionally, (ii) a substance to be delivered.

The pharmaceutical compositions suitable for injectable use in accordance with this invention include sterile aqueous solutions or dispersions and sterile powders or lyopholysates for the extemporaneous preparation of sterile injectable solutions or dispersions. The dosage forms must be sterile and it must be stable under the conditions of manufacture and storage. The carrier for injectable formulations is typically water but can also include ethanol, a polyol (for example, glycerol, propylene glycol and liquid polyethylene glycol), mixtures thereof, and vegetable oil.

Injectable formulations used in the present invention can also be formulated as injectable prolonged release formulations in which the active compound is combined with one or more natural or synthetic biodegradable or biodispersible polymers such as carbohydrates, including starches, gums and etherified or esterified cellulosic derivatives, polyethers, polyesters, polyvinyl alcohols, gelatins, or alginates. Such dosage formulations can be prepared for example in the form of microsphere suspensions, gels, or shaped polymer matrix implants that are well-known in the art for their function as “depot-type” drug delivery systems that provide prolonged release of the biologically active components. Such compositions can be prepared using art-recognized formulation techniques and designed for any of a wide variety of drug release profiles.

One example of a useful formulation which may be used in the methods of the present invention for providing a prolonged duration of action is described in U.S. Pat. No. 7,332,503 (Wikstrom, et al.), hereby incorporated by reference. Therein, apomorphine derivatives and the physiologically acceptable salts thereof as well as formulations thereof are described which provide a prolonged duration of action. The apomorphine pro-drugs can be suspended (as a neat oil or as crystals, or dissolved in a suitable and pharmaceutically acceptable solvent (e.g. water, ethanol, DMSO, i-PrOH or benzylbenzoate)) in a pharmaceutically acceptable depot oil (e.g. viscoleo, sesame oil or olive oil) and injected subcutaneously or intramuscularly with a syringe or a “pen injector”. Alternatively, these drugs may, in a suitable composition and with a suitable vehicle (penetration enhancer), be applied to a patch for transdermal administration. The composition could include also a local anesthetic (e.g. lidocaine) to avoid injection pain, in particular at intramuscular injections. In one embodiment, the composition is in the form of a patch or an ointment for transdermal administration. The patch or ointment preferably also comprises stabilizers, solubilizers and permeation activators to facilitate the passage of the active principle through the skin. In another preferred embodiment, the composition is in the form of a depot preparation for subcutaneous or intramuscular administration comprising an apomorphine derivative or the physiologically acceptable salt thereof dissolved or suspended in an oil. In certain embodiments, in addition to the apomorphine derivative, the formulation further contains a local anesthetic. The formulations described in the '503 patent can be modified as understood by one skilled in the art to contain other active drugs as described herein for use at the BONATH.

An injectable depot formulation is a dosage form, which is generally intended to have a therapeutic activity for 2 to 4 weeks after administration (e.g. neuroleptics like Fluphenazine decanoate in sesame oil). In order to maintain effective drug plasma levels the dosage form should release the drug at a more or less constant rate during the desired dosing interval. The difference between such prior art depots and depots used in the present invention is that the in accordance with the present invention, the drug is not needed to be absorbed into the systemic circulation.

A suitable form of depot preparation is the subcutaneous or intramuscular administration of an oil solution and/or oil suspension of a lipophilic drug. This gives a slow transport over the oil-biofluid interface and a slow dissolution in the biophase. Thus, when the drug is dissolved in a polar solvent (e.g. oils), which is non-miscible with the aqueous biological fluids, the drug has to be transported over the oil/water interface. When the oil/water partition coefficient is high, the transport will be slow. For very lipophilic drugs, the release from the oil phase may last for up to several weeks. The use of depot preparations such as those described herein may be used to deliver the drugs described herein at the BONATH.

The maximum volume of an oil solution/suspension to be injected intramuscularly or subcutaneously is 2-4 mL. This is feasible for the preparations of the aporphine derivatives of the present invention. The accumulated daily dose used in apomorphine s.c. therapy in Parkinson's disease is, e.g., 4-10 times about 1-4 mg (4-40 mg/day). For example, 2 mg Apomorphine HCl (or equivalent molar amount of another dopamine agonist(s), as the base or as a suitable salt or ion-pair) may be dissolved in 1 mL of an oil (sesame oil, Viscoleo or another approved oil) and the mixture gently heated (max 50° C.) shaken in a test tube shaker and ultrasonicated for a short time (minutes) until the mixture becomes a homogeneous solution or suspension. If necessary, the dopamine agonist may first be dissolved in 50-300 .mu.L DMSO, water, t-BuOH, PEG, benzylbenzoate, or another suitable and approved solvent or mixtures thereof, before adding the oil to a total volume of 1 mL.

Another example of a polymeric drug delivery system which may be adapted for use in the present invention by one skilled in the art is described in U.S. Pat. No. 5,601,835 (Sabel, et al.), hereby incorporated by reference, which describes a polymeric drug delivery system for delivery of any substance to the central nervous system. The delivery system is preferably implanted in the central nervous system for delivery of the drug directly to the central nervous system. These implantable devices can be used, for example, to achieve continuous delivery of dopamine, which cannot pass the blood brain barrier, directly into the brain for an extended time period. The implantable devices display controlled, “zero-order” release kinetics, a life time of a minimum of several weeks or months even when the devices contain water soluble, low molecular weight compounds, biocompatibility, and relative non-invasiveness. The polymeric devices are said to be applicable in the treatment of a variety of central nervous system disorders including Parkinson's disease, Alzheimer's dementia, Huntington's disease, epilepsy, trauma, stroke, depression and other types of neurological and psychiatric illnesses, and one skilled in the art can adapt that drug delivery system for delivering the drugs contemplated herein at the BONATH.

Yet another example of a system that may be adapted for use in the present invention is described in U.S. Pat. No. 5,601,835 (Sabel, et al.), hereby incorporated by reference, wherein a compound such as dopamine is encapsulated within a polymer to form a polymeric device, the device formed of a biocompatible polymer that is plastically deformable selected from the group consisting of ethylene vinyl acetate, polyurethanes, polystyrenes, polyamide, polyacrylamide, and combinations thereof having a non-porous polymer coating thereon with one or more openings, with limited water sorptivity and slight permeability to the passage of small, aqueous-soluble molecules, wherein said compound is linearly released (e.g., zero order release) from said polymeric device over a sustained period of time of at least 65 days at a predetermined level and rate when implanted in a patient at a specific site within the central nervous system where the compound is released directly into the central nervous system and the device remains essentially intact throughout the release period. The delivery device is a two-phase system which is manufactured using standard techniques such as blending, mixing or the equivalent thereof, following selection of the biologically active material to be delivered and an appropriate polymer for formation of the matrix. The general method of solvent casting as disclosed by Siegel and Langer, “Controlled release of polypeptides and other macromolecules”, Pharmaceutical Research 1, 2-10 (1984), is modified so that drug is dispersed within the devices to create channels and pores to the surface for release of the drug at the desired rate. Where appropriate, a coating impermeable to the drug is placed over a portion of the drug containing polymer matrix to further regulate the rate of release. One skilled in the art can adapt that drug delivery system for delivering the drugs contemplated herein at the BONATH.

Yet another formulation which may used to deliver the drug (e.g., dopamine agonists) as set forth in the present invention at the BONATH is described in U.S. Pat. No. 7,314,636 (Caseres, et al.), hereby incorporated by reference, which describes injectable implants comprising glycolic acid and bio-compatible/bio-absorbable polymeric particles containing a polymer of lactic acid. The particles are small enough to be injected through a needle but large enough to avoid engulfment by macrophages. The injectables of this invention may be in a pre-activated solid form or an activated form (e.g., injectable suspension or emulsion).

It is further contemplated that the system described in U.S. Pat. No. 6,586,006 (Roser, et al.), hereby incorporated by reference, can be adapted by one skilled in the art for use in the present invention for delivery of drugs at the BONATH. Therein are described delivery systems suitable for delivery of bioactive materials to subcutaneous and intradermal, intramuscular, intravenous tissue, the delivery system being sized and shaped for penetrating the epidermis. The delivery systems comprises a vitreous vehicle loaded with the guest substance and capable of releasing the guest substance in situ at various controlled rates. Subdermal implantable therapeutic systems have also been formulated for slow release of certain pharmaceutical agents for extended periods of time such as months or years. A well-known example is Norplant® for delivery of steroid hormones.

In membrane permeation-type controlled drug delivery, the drug is encapsulated within a compartment that is enclosed by a rate-limiting polymeric membrane. The drug reservoir may contain either drug particles or a dispersion (or solution) of solid drug in a liquid or a matrix type dispersing medium. The polymeric membrane may be fabricated from a homogeneous or a heterogeneous nonporous polymeric material or a microporous or semipermeable membrane. The encapsulation of the drug reservoir inside the polymeric membrane may be accomplished by molding, encapsulation, microencapsulation, or other techniques. The implants release drugs by dissolution of the drug in the inner core and slow diffusion across the outer matrix. The drug release from this type of implantable therapeutic system should be relatively constant and is largely dependent on the dissolution rate of the drug in the polymeric membrane or the diffusion rate across or a microporous or semipermeable membrane. The inner core may substantially dissolve over time; however, in devices currently in use, the outer matrix does not dissolve.

Other implantable therapeutic systems involve matrix diffusion-type controlled drug delivery. The drug reservoir is formed by the homogeneous dispersion of drug particles throughout a lipophilic or hydrophilic polymer matrix. The dispersion of drug particles in the polymer matrix may be accomplished by blending the drug with a viscous liquid polymer or a semisolid polymer at room temperature, followed by cross-linking of the polymer, or by mixing the drug particles with a melted polymer at an elevated temperature. It can also be fabricated by dissolving the drug particles and/or the polymer in an organic solvent followed by mixing and evaporation of the solvent in a mold at an elevated temperature or under vacuum. The rate of drug release from this type of delivery device is not constant. Examples of this type of implantable therapeutic system are the contraceptive vaginal ring and Compudose implant. PCT/GB 90/00497 describes slow release glassy systems for formation of implantable devices. The described implants are bioabsorbable and need not be surgically removed. One skilled in the art can adapt these drug delivery systems for delivering the drugs contemplated herein at the BONATH.

In microreservoir dissolution-controlled drug delivery, the drug reservoir, which is a suspension of drug particles in an aqueous solution of a water-miscible polymer, forms a homogeneous dispersion of a multitude of discrete, unleachable, microscopic drug reservoirs in a polymer matrix. The microdispersion may be generated by using a high-energy-dispersing technique. Release of the drug from this type of drug delivery device follows either an interfacial partition or a matrix diffusion-controlled process. An example of this type of drug delivery device is the Syncro-Mate-C Implant.

Yet another formulation which may be adapted by one skilled in the art for use in the present invention is described in U.S. Pat. No. 6,576,263 (Truong, et al.), hereby incorporated by reference, which describes a preformed object for delivering an active agent for a subject, the preformed object including crosslinked protein, and methods of making and using.

Yet another formulation which may be adapted by one skilled in the art for use in the present invention is described in U.S. Pat. No. 6,287,588 (Shih, et al.), hereby incorporated by reference, which describes a composition and method for releasing a bio-active agent or a drug within a biological environment in a controlled manner. The composition is a dual phase polymeric agent-delivery composition comprising a continuous biocompatible gel phase, a discontinuous particulate phase comprising defined microparticles and an agent to be delivered. A microparticle containing a bio-active agent is releasably entrained within a biocompatible polymeric gel matrix. The bio-active agent release may be contained in the microparticle phase alone or in both the microparticles and the gel matrix. The release of the agent is prolonged over a period of time, and the delivery may be modulated and/or controlled. In addition, a second agent may be loaded in some of the microparticles and/or the gel matrix.

Yet another formulation which may be adapted by one skilled in the art for use in the present invention is described in U.S. Pat. No. 7,364,568 (Angel, et al.), hereby incorporated by reference, which describes a transdermal transport device includes a reservoir for holding a formulation of an active principle, and a needle with a bore extending along the length of the needle from a first end of the needle to a second end of the needle. The second end is substantially aligned to a plane parallel to a body surface of a biological body when the device is placed on the body surface. The device also includes an actuator which pumps the formulation through the bore of the needle between a target area of the body and the reservoir.

In yet other embodiments of the invention, the dopamine agonist is infused into the patient at the site of the injury using technology known to be useful for infusing other drugs, such as an insulin pump. One such system, U.S. Pat. No. 7,354,420 (Steil, et al.), hereby incorporated by reference, describes a closed loop infusion system controls the rate that fluid is infused into the body of a user. The closed loop infusion system includes a sensor system, a controller, and a delivery system. The sensor system includes a sensor for monitoring a condition of the user. The sensor produces a sensor signal, which is representative of the condition of the user. The sensor signal is used to generate a controller input. The controller uses the controller input to generate commands to operate the delivery system. The delivery system infuses a liquid into the user at a rate dictated by the commands from the controller. Preferably, the sensor system monitors the glucose concentration in the body of the user, and the liquid infused by the delivery system into the body of the user includes insulin.

The present invention is contemplated to encompass all implantable or injectable formulations, e.g., the technologies described above, with the inclusion of a drug(s) (e.g., dopamine agonist(s)), such that the administration of a drug useful for treatment of disease state or condition in humans via topical brainstem afferent stimulation (de-afferentation) therapy. Therefore, modifications of the invention via, e.g., the choice and/or amount of drug are considered to be obvious variations of this disclosure and within the scope of the appended claims.

### DETAILED DESCRIPTION OF PREFERRED EMBODIMENTS

The present invention will now be more fully described with reference to the accompanying examples. It should be understood, however, that the following description is illustrative only and should not be taken in any way as a restriction on the generality of the invention specified above.

### Example 1

### Topical Formulation

An aqueous based apomorphine cream was produced using Lipoderm® as the carrier. Lipoderm®/LIP is a commercially marketed compounding agent (from PCCA, Pharmaceutical Compounding Centers of America) having the following ingredients: Ethoxydiglycol, Water (Aqua), Glycerin, C12-15Alkyl Benzoate, Glyceryl Stearate, Dimethicone, Cetearyl Alcohol, Cetearyl Glucoside, Polyacrylamide, Cetyl Alcohol, Magnesium Aluminum Silicate, Xanthan Gum, Aloe Vera (Aloe Barbadensis), Tocopheryl Acetate (Vitamin E Acetate), Prunus Amygadalus Amara (Bitter Almond) Kernel Oil, Vitis Vinifera (Grape) Seed Extract, Triticum Vulgare (Wheat) Germ Oil, Retinyl Palmitate (Vitamin A Palmitate), Ascorbyl Palmitate (Vitamin C Palmitate), Pro-Lipo Multi-emulsion Liposomic System, Tetrasodium EDTA, Phenoxyethanol, and Sodium Hydroxymethylglycinate. The concentration was 1 mg of apomorphine in 1 ml of Lipoderm. The concentration of apomorphine in the Lipoderm is 1 mg in 0.5 ml. Lipoderm is a whitish cream with no smell.

### Example 2

### Topical Formulation

An aqueous based tizanidine cream was produced using Lipoderm® as the carrier. Lipoderm®/LIP is a commercially marketed compounding agent (from PCCA, Pharmaceutical Compounding Centers of America) having the following ingredients: Ethoxydiglycol, Water (Aqua), Glycerin, C12-15Alkyl Benzoate, Glyceryl Stearate, Dimethicone, Cetearyl Alcohol, Cetearyl Glucoside, Polyacrylamide, Cetyl Alcohol, Magnesium Aluminum Silicate, Xanthan Gum, Aloe Vera (Aloe Barbadensis), Tocopheryl Acetate (Vitamin E Acetate), Prunus Amygadalus Amara (Bitter Almond) Kernel Oil, Vitis Vinifera (Grape) Seed Extract, Triticum Vulgare (Wheat) Germ Oil, Retinyl Palmitate (Vitamin A Palmitate), Ascorbyl Palmitate (Vitamin C Palmitate), Pro-Lipo Multi-emulsion Liposomic System, Tetrasodium EDTA, Phenoxyethanol, and Sodium Hydroxymethylglycinate. Lipoderm is a whitish cream with no smell.

### Example 3

The efficacy of a topical apomorphine formulation was studied in human patients. For each of these patients, an amount of the compounded cream prepared in accordance with Example 1.

Generally the drug applications have been over areas of nerve entrapment (tarsal and carpal tunnel syndromes) or over the affected muscle and its insertion points.

Similar to TRNA at BONATH, all patients have reported pain relief within 5-10 minutes. Two patients were marathon runners who were able to run events within a week of several treatments. Before treatment, they were both experiencing pain and disability to the extent walking was difficult. Testimonials of two such patients are attached herewith.

From the above observations, it is apparent TRNA may be applied for peripheral nerve injury states with associated pain and muscle spasm. The symptoms may be from localized phenomena in addition to that of afferent feed-back to CNS sensory processing areas with efferent outflow back to the site of injury and other regions.

This possibility was first observed several years ago in a female patient who, after a stroke, was left with a spastic upper extremity. It was contractured to a useless state. She was elderly and intolerant of the usual oral anti-spasmodic medications tizanidine, baclofen, and the benzodiazepines, such as Valium and Klonopin. Topical tizanidine was applied to the extensor forearm muscles 1 mg twice daily. Within several weeks, visible improvement was noted. After a few months of continued therapy, the extremity, which had been in a state of disuse for nearly 2 years, was essentially back to normal. The patient was able to return to her love for cooking.

The single dose applications of topical apomorphine for the treatment of neuropathic pain have been in the range of 0.5-1 mg. This has been increased to 2 mg in some instances without deleterious effects, likely as drug application is peripheral, as opposed to central, as at BONATH for CNS related disorders.

### Example 4

The topical tizanidine single dose application for peripheral manifestations of muscle spasm and spasticity have been in the 1-2 mg range and could be increased to 4-6 mg for the same reasons.

### Example 5

Several conditions have been successfully treated with application of compounded drug topically to the site of injury. These conditions have consisted of tarsal tunnel syndrome/plantar fasciitis in distance runners, localized foot pain from stress fractures, carpal tunnel syndrome, hamstring injuries, and acute lumbar sprain with and without radiating (radicular) component. These treatments were conducted with apomorphine 0.5 mg/ml in Lipoderm.
1. Carpal tunnel syndrome: 20 yr. old pregnant female with 0.5 mg application without recurrent symptoms. Compounded drug was applied at the wrist—carpal tunnel.
2. Tarsal tunnel syndrome/plantar fasciaitis: 33 yr. female runner with 90% pain relief with 0.5 mg application, followed by additional 0.5 mg after 24 hours with complete pain relief and full return of function to running a marathon. Drug was applied at the tarsal tunnel and over the Abductor Hallucis muscle. Patient had prior local steroid injection and immobilization of the foot without benefit.
3. Exacerbation of prior hamstring injury: 60 yr. old male with 6-8 hours of discomfort with inability to sleep. 5-10 minutes after 0.5 mg to the muscle and insertion site, complete resolution of pain without recurrence.
4. Acute lumbar sprain unresponsive to over-the-counter NSAIDS and Ultram: 48 yr. old female after 1.0 mg application experienced 80-90% pain and spasm relief within 10-15 minutes that lasted overnight. The next day, re-applied 0.5 mg for recurrent discomfort at a much lower level with again relief. Patient had not experienced relief of this nature in the past with steroids and narcotics.
5. Acute lumbar sprain from lifting: 20 yr. old female responded to 0.5 mg application and placement of a heating pad to area without recurrent symptoms.
6. Foot pain related to stress fractures and tarsal tunnel in competitive marathon runner: 35 yr. old female with pain for several weeks experienced pain relief after application of 0.5 mg to the base of toe of fractures and 0.5 to the tarsal tunnel. She was able to compete in the Boston Marathon 2 weeks later.

### Example 6

The efficacy of topical opioid analgesic formulations was studied in human patients. For each of these patients, a specified dose of an opioid analgesic as described below in the compounded cream prepared in accordance with Example 1 is applied to the skin of the human patient as follows:

Patient A: A 66 year-old female s/p lumbar laminectomy, post-polio syndrome, and ischemic stroke with chronic neuropathic pain and failed back syndrome. Her routine medications for pain consisted of hydrocodone 10/500 (10 mg hydrocodone/500 mg acetaminophen) three times per day and tramadol 50-100 mg every 4 hours. Responded to initial therapy with topical morphine sulfate 2.5 mg/0.5 ml of proprietary compounding medium, “Pass-gel” through Mike Pass of Family Pharmacy, Sarasota, Fla. Patient achieved significant relief of neck and back pain within 15 minutes which lasted several hours. She was begun on a daily regimen of topical morphine sulfate (MS) 2.5 mg to the nuchal region and 2.5 mg to the back at the upper part of the surgical site at the midline and instructed to taper off narcotics. After one month, she was off narcotics completely but developed a rash to “Pass-gel” and switched to topical tramadol in Lipoderm (40 mg tramadol hydrochloride dose) twice daily to be applied in the same fashion. She is now doing well and off oral narcotics and tramadol tablets. Pain relief is achieved with the topical tramadol treatment twice daily. Her cognitive functioning and quality of life is improved. She has been on the regimen 6 weeks.

Patient B: A 70 year-old female with M.S., rheumatoid arthritis, fibromyalgia and associated chronic pain syndrome on Percocet® 5/325: two-three times per day. Had bilateral facet blocks, SI joint injections and trigger point injections with minimal short-lived relief. Patient presented with about 7-8/10 pain in the neck and about 7/10 pain in the back and legs. 15 minutes after 20 mg of topical tramadol (Tramo-Top) to the nuchal region and to the lower back, her pain decreased to about 0/10 in the neck and about 2/10 in the back and legs. There was marked associated improved range of motion and function. Her usual improvement with Percocet® is from about 7-8 to about 5-6 after about an hour. She was begun on a regimen of topical tramadol 20-40 mg to the nuchal region and back twice a day as needed with instructions to taper off Percocet. Patient B had been on tramadol (Ultram) tablets in the past without significant benefit from them.

Patient C: A 74 year-old male with post-herpetic neuralgia, fibromyalgia and chronic arthritis came with severe right knee pain of two week's duration. He had seen an orthopedic surgeon who had injected the knee with minimal effect. He was told he would need a knee replacement. Application of 40 mg of topical tramadol divided between the knee (popliteal fossa) and the lower back resulted in initial (approximately) 50% reduction in pain within 15 minutes. This eventually reached about 80-90% relief with marked improvement in mobility. Patient is now using it as needed. He had been intolerant of oxycodone and Percocet® in the past.

Patient D: A 58 year-old female with chronic pain syndrome from fall with neck injury, headaches, and lumbar radiculopathy who has been on long-term narcotics—Demerol® for 10 years and most recently, hydromorphone. She responded to initial therapy with topical morphine 2.5 mg in 0.5 ml “Pass-gel” with additional about 20-30% pain relief despite taking hydromorphone on a regular basis. She has been topically administered (nuchal) morphine sulfate 2.5 mg twice daily for several weeks in accordance with the present invention with benefit to the extent she has been able to reduce the oral hydromorphone use. Most recently, she was tried 40 mg of topical tramadol at the nuchal region with benefit. She has now been converted to using topical tramadol 40 mg twice daily in place of morphine sulfate and instructed to continue the tapering process off hydromorphone.

Patient E: A 48 year-old male with history of cervical, thoracic and lower back injuries from hard labor, developed fibromyalgia and chronic pain syndrome requiring daily use of oral dosages of hydrocodone, Cymbalta®, and Lyrica®. The patient was recently treated with 40 mg of topical tramadol applied to the neck and back in divided dose with about 70-80% reduction in pain at the various above places within about 10-15 minutes. Because of his desire to be off narcotics as they were causing lethargy and fatigue that was interfering with his work, he has been switched to topical tramadol one to two times per day as needed in place of oral hydromorphone.

Patient F: A 61 year-old male with cervical radiculopathy, bilateral carpal tunnel syndromes, lumbar degenerative disc disease, and s/p recent ankle surgery, is on oral tramadol tablets 50 mg: 4-5×/day as well as venlafaxine, lamotrigene, and Lyrica®. The patient was treated with 40 mg of topical tramadol to the nuchal region and lower back in divided doses and achieved 70+% pain relief within about 15 minutes. He has been switched from oral tramadol to topical tramadol once to twice daily.

Patient G: A 44 year-old female with failed back syndrome from back operations with chronic pain and narcotic drug dependency, using oral oxycodone 30 mg three-four times per day. Patient G had 3 mg of topical morphine sulfate in “Pass-gel” applied to the nuchal region with about 20-30% further reduction in pain from baseline which lasted several hours.

Patient H: A 42 year-old female with 9 month use of oxycodone 3-4×/day for treatment of back and leg pain after injury and surgery of the ankle. Patient desired coming off narcotics as they were interfering with her quality of life with husband and 3 children. 40 mg of topical tramadol in 1 ml Lipoderm was applied to the neck and back in divided doses. She experienced greater than 80% pain relief within about 15 minutes. This improved further and lasted nearly 48 hours, during which time she did not take narcotics.

Patient I: A 57 year-old female s/p auto accident with head injury and associated neck and back injuries and chronic pain, 12 year history of narcotics use as roxicodone 6×/day. With the application of topical morphine sulfate 2.5 mg in 0.5 ml proprietary “Pass-gel,” she experienced about 15-20% reduction in her baseline back and hip pain present despite regular use of narcotics.

Patient J: A 42 year-old male, s/p glass explosion injury to the right face with trigeminal neuralgia and cervical sprain, incapacitating in nature, experienced dramatic improvement in pain with the application of topical tramadol 40 mg in 1 ml Lipoderm to the nuchal region. He had failed Trileptal®, Lyrica®, Neurontin®, Savella®, and nerve and stellate ganglion blocks. He is now on topical tramadol 40 mg once to twice daily.

Patient K: A 53 year-old female with cervical, thoracic, and lumbar spondylosis and migraines, has been on chronic narcotic use for several years in the form of oxycodone four to six times per day and MS Contin® 30 mg twice daily. With the application of topical morphine sulfate 2.5 mg to the nuchal region, she experienced reduction in headache and neck pain from “severe” to 0-1 of 10 within 10-15 minutes of application which lasted several hours.

Patient L: A 76 year-old male with cervical injury and fusion, on Fentanyl® pain patch 50 ug/hr, tramadol tablets 50 mg four times per day, and oxycodone 5 mg tablets, one-two doses every 8 hours. Topical morphine sulfate 2.5 mg was applied to the nuchal region and pain was reduced from about 8 of 10 to about 4 of 10 within 8-10 minutes which lasted for the rest of the day.

### CONCLUSION

The examples provided above are not meant to be exclusive. Many other variations of the present invention would be obvious to those skilled in the art, and are contemplated to be within the scope of the appended claims.

The hypotheses of the inventor provided throughout the specification are for possible explanation purposes only, and are not meant to be limiting in any way.

1. A method of treating peripheral neuropathic pain in humans resulting from a peripheral nerve injury or muscle spasm resulting from a peripheral nerve injury in humans comprising administering a pharmaceutical formulation comprising a therapeutically effective amount of a drug selected from the group consisting of a dopamine agonist, a skeletal muscle relaxant, an opioid agonist, a SNRI (serotonin-norepinephrine reuptake inhibitor), and a combination thereof topically at the site of the injury.

2. The method of claim 1, wherein the injury is neuronal hyperexcitability and/or a neurochemical dysfunction syndrome.

3. The method of claim 1, wherein the formulation is applied topically over an over the affected muscle and its insertion points.

4. The method of claim 1, wherein the formulation is applied topically over an area of nerve entrapment (tarsal and carpal tunnel syndromes).

5. The method of claim 1, wherein the drug is a dopamine agonist.

6. The method of claim 5, wherein the dopamine agonist is selected from the group consisting of apomorphine, pramipexole, ropinirole, bromocriptine, cabergoline, pergolide, rotigotine, entacapone, tocapone, seligiline, and mixtures of any of the foregoing.

7. (canceled)

8. The method of claim 6, wherein the dopamine agonist is apomorphine and the single dose application of topical apomorphine for the treatment of neuropathic pain is in the range from about 0.5 to about 2 mg.

9. (canceled)

10. The method of claim 1, wherein the drug is a skeletal muscle relaxant.

11. The method of claim 10, wherein the drug is selected from the group consisting of afloqulone, baclofen, botulin toxins, carisoprodol, chlormezanone, chlorphenesin carbamate, chlorzoxazone, cyclobenzaprine, clonazepam, dantrolene, diazepam, eperisone, idrocilamide, inaperisone, mephenesin, mephenoxalone, methocarbamol, metaxalone, mivacurium chloride, orphenadrine, phenprobamate, pridinol mesylate, quinine, tetrazepam, thiocolchicoside, tizanidine, tolperisone, pharmaceutically acceptable salts thereof, active metabolites thereof, prodrugs thereof and mixtures thereof.

12. The method of claim 11, wherein the skeletal muscle relaxant is tizanidine base, tizanidine hydrochloride or any pharmaceutically acceptable salts thereof, prodrugs thereof or mixtures thereof in the range from about 1 to about 6 mg per application.

13-16. (canceled)

17. The method of claim 16, wherein the drug is an opioid agonist is selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tramadol, salts of any of the foregoing, and mixtures of any of the foregoing.

18. The method of claim 17, wherein the opioid agonist is tramadol in an amount from about 20 mg to about 40 mg.

19. The method of claim 17, wherein the opioid agonist is morphine sulfate in an amount from about 2.5 to about 5 mg.

20. The method of claim 1, wherein the drug is formulated in a pharmaceutically acceptable immediate release topical carrier which is an aqueous-based gel or cream.

21-26. (canceled)

27. The method of claim 1, wherein the drug is a dopamine agonist in a sustained release delivery system capable of delivering from about 0.25 mg to about 6.0 mg of the dopamine agonist through the skin of a human patient over a 24 hour period, the transdermal delivery system being capable of delivering the dopamine agonist in such amounts for a time period from about 1 to about 7 days.

28. (canceled)

29-34. (canceled)

35. The method of claim 1, wherein the drug is a skeletal muscle relaxant in a sustained release delivery system is capable of delivering from about 0.25 mg to about 6.0 mg of the skeletal muscle relaxant through the skin of a human patient over a 24 hour period, the transdermal delivery system being capable of delivering the skeletal muscle relaxant in such amounts for a time period from about 1 to about 7 days.

36. The method of claim 1, wherein the drug is tramadol in a sustained release delivery system is capable of delivering from about 20 mg to about 80 mg of the tramadol through the skin of a human patient over a 24 hour period, the transdermal delivery system being capable of delivering the tramadol in such amounts for a time period from about 1 to about 7 days.

37. The method of claim 1, further comprising by applying a therapeutically effective amount of a drug selected from the group consisting of a dopamine agonist, a skeletal muscle relaxant, an opioid agonist, an SNRI, and any combination thereof at two or more sites along the nerve leading from the site of the injury to the central nervous system.

38. The method of claim 37, wherein the drug(s) is applied at the Median or Ulnar Nerve at the wrist and on the arm, and elbow.

39. The method of claim 37, wherein the drug(s) is applied at tibial nerve at the ankle.

40. The method of claim 37, wherein the drug(s) is applied at the Peroneal Nerve at the fibula head and at the popliteal fossa at the knee.

41. (canceled)
